SEQUENCE LISTING (56 sequences)

SEQ ID NO: 1 - protein, 118 aa, Homo sapiens
VQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARRHYDFDAFDIWGQGTMVTVSS

SEQ ID NO: 2 - protein, 111 aa, Homo sapiens
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTLYVFGTGTKVTVL

SEQ ID NO: 3 - protein, 120 aa, Homo sapiens
QVQLQESGPGLVKPSGTLSLTCAVSGGSISSSNWWSWVRQPPGKGLEWIGEIYHSGNTNYNPSLKSRVTVSVDKSKNQFSLKLTSVTAADTAVYYCARDVSGGVNWFDPWGQGTLVTVSS

SEQ ID NO: 4 - protein, 110 aa, Homo sapiens
NFMLTQPHSVSESPGKTVTISCTRSSGSIASNYVQWYQQRPGSAPTTVIYEDNQRPSGVPDRFSGSIDSSSNSASLTISGLKTEDEADYYCQSYDSSTVVFGGGTKLTVL

SEQ ID NO: 5 - protein, 114 aa, Homo sapiens
VQLVQSGAEAKKPGESLKISCKASGYSFTTFWIGWVRQMPGSGLEWIGIIYPGDSDTRYSPSFQGHVTISADRSTSTAYLQWSSLKASDTAMYYCARSAVFDYWGQGTLVTVSS

SEQ ID NO: 6 - protein, 109 aa, Homo sapiens
EIVMTQSPATLSVSPGGRATLSCRASQSVSSNLAWYQQKPGQAPRLLIYGASTRATGIPVRFSGSGSGTEFTLTISSLQSEDFAVYYCQQYNNWPTSWTFGQGTKVEIK

SEQ ID NO: 7 - protein, 118 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDYGDYYFDYWGQGTLVTVSS

SEQ ID NO: 8 - protein, 110 aa, Homo sapiens
QSALTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTGVFGEGTKLTVL

SEQ ID NO: 9 - protein, 120 aa, Homo sapiens
EVQLVESGGGVGRPGGSLRLSCAASGFTFDDYGMSWVRQVPGKGLEWVSGINWSGADTGYADSVKGRFTISRDNAKNSLYLQMNSLRVEDTALYHCAREVGIAVTGYWFDPWGQGTLVTV

SEQ ID NO: 10 - protein, 108 aa, Homo sapiens
SYELTQPPSVSVAPGQTARISCGGNHSESVHWYQQKPGQAPVLVVYDDSDRPSGIPERFSGSKSGNTATLTISRVEGGDEAEYYCQVWDSSSDHPGVVFGGGTKLTVL

SEQ ID NO: 11 - protein, 118 aa, Homo sapiens; identical to SEQ ID NO: 7

SEQ ID NO: 12 - protein, 107 aa, Homo sapiens
LTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTGVFGGGTKLTVL

SEQ ID NO: 13 - protein, 120 aa, Homo sapiens
EVQLVESGGGLVQPGRSLRLSCAASGFTFDDYAMHWVRQVPGKGLEWVSGISWSSGSIGYADSVKGRFTISRDNAKNSLYLQMNSLRAEDTALYYCAKDRASSWYAYGMDVWGQGTLVTV

SEQ ID NO: 14 - protein, 106 aa, Homo sapiens
NFMLTQPHSVSESPGKTVTISCTRSSGSIASNYVQWYQQRPGSSPTTVIYDDNQRPSGVPNRFSGSIDSSSNSASLIISGLKTEDEADYYCQSTRVFGGGTKLTVL

SEQ ID NO: 15 - protein, 8 aa, Homo sapiens
GFTFSSYG

SEQ ID NO: 16 - protein, 8 aa, Homo sapiens
IWYDGSNK

SEQ ID NO: 17 - protein, 12 aa, Homo sapiens
ARRHYDFDAFDI

SEQ ID NO: 18 - protein, 9 aa, Homo sapiens
SSDVGGYNY

SEQ ID NO: 19 - protein, 3 aa, Homo sapiens
EVS

SEQ ID NO: 20 - protein, 11 aa, Homo sapiens
SSYTSSSTLYV

SEQ ID NO: 21 - protein, 9 aa, Homo sapiens
GGSISSSNW

SEQ ID NO: 22 - protein, 7 aa, Homo sapiens
IYHSGNT

SEQ ID NO: 23 - protein, 13 aa, Homo sapiens
ARDVSGGVNWFDP

SEQ ID NO: 24 - protein, 8 aa, Homo sapiens
SGSIASNY

SEQ ID NO: 25 - protein, 3 aa, Homo sapiens
EDN

SEQ ID NO: 26 - protein, 9 aa, Homo sapiens
QSYDSSTVV

SEQ ID NO: 27 - protein, 8 aa, Homo sapiens
GYSFTTFW

SEQ ID NO: 28 - protein, 8 aa, Homo sapiens
IYPGDSDT

SEQ ID NO: 29 - protein, 8 aa, Homo sapiens
ARSAVFDY

SEQ ID NO: 30 - protein, 6 aa, Homo sapiens
QSVSSN

SEQ ID NO: 31 - protein, 3 aa, Homo sapiens
GAS

SEQ ID NO: 32 - protein, 11 aa, Homo sapiens
QQYNNWPTSWT

SEQ ID NO: 33 - protein, 8 aa, Homo sapiens
GYSFTSYW

SEQ ID NO: 34 - protein, 7 aa, Homo sapiens
IYPGDSD

SEQ ID NO: 35 - protein, 11 aa, Homo sapiens
ARDYGDYYFDY

SEQ ID NO: 36 - protein, 9 aa, Homo sapiens
SSDVGGYNY

SEQ ID NO: 37 - protein, 3 aa, Homo sapiens
DVS

SEQ ID NO: 38 - protein, 10 aa, Homo sapiens
CSYAGSYTGV

SEQ ID NO: 39 - protein, 8 aa, Homo sapiens
GFTFDDYG

SEQ ID NO: 40 - protein, 8 aa, Homo sapiens
INWSGADT

SEQ ID NO: 41 - protein, 15 aa, Homo sapiens
AREVGIAVTGYWFDP

SEQ ID NO: 42 - protein, 4 aa, Homo sapiens
HSES

SEQ ID NO: 43 - protein, 3 aa, Homo sapiens
DDS

SEQ ID NO: 44 - protein, 13 aa, Homo sapiens
QVWDSSSDHPGVV

SEQ ID NO: 45 - protein, 8 aa, Homo sapiens
GYSFTSYW

SEQ ID NO: 46 - protein, 8 aa, Homo sapiens
IYPGDSDT

SEQ ID NO: 47 - protein, 11 aa, Homo sapiens
ARDYGDYYFDY

SEQ ID NO: 48 - protein, 9 aa, Homo sapiens
SSDVGGYNY

SEQ ID NO: 49 - protein, 3 aa, Homo sapiens
DVS

SEQ ID NO: 50 - protein, 10 aa, Homo sapiens
CSYAGSYTGV

SEQ ID NO: 51 - protein, 8 aa, Homo sapiens
GFTFDDYA

SEQ ID NO: 52 - protein, 8 aa, Homo sapiens
ISWSSGSI

SEQ ID NO: 53 - protein, 15 aa, Homo sapiens
AKDRASSWYAYGMDV

SEQ ID NO: 54 - protein, 8 aa, Homo sapiens
SGSIASNY

SEQ ID NO: 55 - protein, 3 aa, Homo sapiens
DDN

SEQ ID NO: 56 - protein, 5 aa, Homo sapiens
QSTRV

(19) United States
(12) Patent Application Publication
(10) Pub. No.: US 2018/0244802 A1
(43) Pub. Date: Aug. 30, 2018

(54) ANTIBODY OR ANTIBODY FRAGMENT OF NON-IG SCAFFOLD BINDING TO A BINDING REGION OF AN ANTI-N-METHYL-D-ASPARTATE (NMDA) RECEPTOR ANTIBODY

(71) Applicant: DEUTSCHES ZENTRUM FUR NEURODEGENERATIVE ERKRANKUNGEN E.V. (DZNE), Bonn (DE)

(72) Inventor: Harald PRUESS, Berlin (DE)

(73) Assignee: DEUTSCHES ZENTRUM FUR NEURODEGENERATIVE ERKRANKUNGEN E.V. (DZNE), Bonn (DE)

(21) Appl. No.: 15/753,642

(22) PCT Filed: Aug. 16, 2016
(86) PCT No.: PCT/EP2016/069451

§ 371 Date: Feb. 20, 2018

(30) Foreign Application Priority Data
Aug. 17, 2015 (EP) 15181290.6

(51) Int. Cl. C07K 16/42; C07K 16/28; G01N 33/68; G01N 33/53; C12N 15/115

(52) U.S. Cl. CPC C07K 16/4208; C07K 16/286; G01N 33/6854; G01N 33/5308; C12N 2310/16; C07K 2317/24; C07K 2317/76; C07K 2317/92; G01N 2333/70571; C12N 15/115

(57) ABSTRACT

Subject matter of the present invention is an Antibody or antibody fragment or non-Ig scaffold binding to a binding region of an anti-NMDAR1 antibody and its uses in therapy or diagnosis.

Subject matter of the present invention is an Antibody or antibody fragment or non-Ig scaffold binding to a binding region of an anti-N-methyl-D-aspartate (NMDA) receptor-1 antibody and its uses in therapy and diagnostics.

Patients with anti-N-methyl-D-aspartate (NMDA) receptor-1 (anti-NMDAR1) antibody encephalitis suffer from a severe form of encephalitis with characteristic clinical multistage features, predominantly affecting children and young women. It progresses from psychiatric symptoms, memory deficits, and epileptic seizures into a state of loss of consciousness, autonomic dysfunction, dyskinesias and hypoventilation (Dalmau et al. 2011. Lancet Neurol. 10(1):63-74, Prüss et al. 2010. Neurology. 75(19):1735-9; Prüss et al. 2013. Neurology. 78(22):1743-53.). Hallmark of the disease are antibodies against the NR1 subunit of the NMDAR1. In addition, a subgroup of patients with atypical dementia harbors anti-NMDAR1 antibodies, removal of which by unspecific removal of all antibodies resulted in clinical improvement in selected cases (Prüss et al. 2010. Neurology. 75(19):1735-9, Doss et al. 2014. Ann Clin Transl Neurol. 1(10):822-32). This has profoundly changed the therapeutic concept in encephalitis as a disease that until now had no causal treatment options. Antibody-mediated diseases are up to date only treatable using aggressive and unspecific immunotherapy including plasma exchange, which implies major side effects by the broad suppression of the immune system, such as frequent infections or sepsis, absent responses to vaccinations, allergic reactions, and cardiovascular complications from central catheters. So far, a specific therapy targeting only the disease-causing autoantibodies against NMDAR1 is urgently needed but not available.

Therefore, the aim of the present approach is to develop a novel antibody-specific immunotherapy that depletes essentially only anti-NMDAR1 antibodies, leaving essentially all other types of ‘beneficial’ antibodies unaffected.

Subject matter of the present invention is an antibody or antibody fragment or non-Ig scaffold binding to a binding region of an anti-NMDAR1 antibody wherein the binding region of said anti-NMDAR1 antibody is comprised in a sequence that is selected from a group consisting of the following sequences:

(003-109-HC)
SEQ ID NO: 1
VQLVESGGGVVQPGRSLRLSCAASGFTFSSYGMHWVRQAPGKGLEWVAV
IWYDGSNKYYADSVKGRFTISRDNSKNTLYLQMNSLRAEDTAVYYCARR
HYDFDAFDIWGQGTMVTVSS
(003-109-LC)
SEQ ID NO: 2
QSALTQPASVSGSPGQSITISCTGTSSDVGGYNYVSWYQQHPGKAPKLM
IYEVSNRPSGVSNRFSGSKSGNTASLTISGLQAEDEADYYCSSYTSSST
LYVFGTGTKVTVL
(003-102-HC)
SEQ ID NO: 3
QVQLQESGPGLVKPSGTLSLTCAVSGGSISSSNWWSWVRQPPGKGLEWI
GEIYHSGNTNYNPSLKSRVTVSVDKSKNQFSLKLTSVTAADTAVYYCAR
DVSGGVNWFDPWGQGTLVTVSS
(003-102-LC)
SEQ ID NO: 4
NFMLTQPHSVSESPGKTVTISCTRSSGSIASNYVQWYQQRPGSAPTTVI
YEDNQRPSGVPDRFSGSIDSSSNSASLTISGLKTEDEADYYCQSYDSST
VVFGGGTKLTVL
(007-168-HC)
SEQ ID NO: 5
VQLVQSGAEAKKPGESLKISCKASGYSFTTFWIGWVRQMPGSGLEWIGI
IYPGDSDTRYSPSFQGHVTISADRSTSTAYLQWSSLKASDTAMYYCARS
AVFDYWGQGTLVTVSS
(007-168-LC)
SEQ ID NO: 6
EIVMTQSPATLSVSPGGRATLSCRASQSVSSNLAWYQQKPGQAPRLLIY
GASTRATGIPVRFSGSGSGTEFTLTISSLQSEDFAVYYCQQYNNWPTSW
TFGQGTKVEIK
(007-169-HC)
SEQ ID NO: 7
EVQLVQSGAEVKKPGESLKISCKGSGYSFTSYWIGWVRQMPGKGLEWMG
IIYPGDSDTRYSPSFQGQVTISADKSISTAYLQWSSLKASDTAMYYCAR
DYGDYYFDYWGQGTLVTVSS
(007-169-LC)
SEQ ID NO: 8
QSALTQPRSVSGSPGQSVTISCTGTSSDVGGYNYVSWYQQHPGKAPKLM
IYDVSKRPSGVPDRFSGSKSGNTASLTISGLQAEDEADYYCCSYAGSYT
GVFGEGTKLTVL
(007-124-HC)
SEQ ID NO: 9
EVQLVESGGGVGRPGGSLRLSCAASGFTFDDYGMSWVRQVPGKGLEWVS
GINWSGADTGYADSVKGRFTISRDNAKNSLYLQMNSLRVEDTALYHCAR
EVGIAVTGYWFDPWGQGTLVTV
(007-124-LC)
SEQ ID NO: 10
SYELTQPPSVSVAPGQTARISCGGNHSESVHWYQQKPGQAPVLVVYDDS
DRPSGIPERFSGSKSGNTATLTISRVEGGDEAEYYCQVWDSSSDHPGVV
FGGGTKLTVL
(007-142-HC)
SEQ ID NO: 11
EVQLVQSGAEVKKPGESLKISCKGSGYSFTSYWIGWVRQMPGKGLEWMG
IIYPGDSDTRYSPSFQGQVTISADKSISTAYLQWSSLKASDTAMYYCAR
DYGDYYFDYWGQGTLVTVSS
(007-142-LC)
SEQ ID NO: 12
LTQPRSVSGSPGQSVTISCTGTSSDVGGYNYVSWYQQHPGKAPKLMIYD
VSKRPSGVPDRFSGSKSGNTASLTISGLQAEDEADYYCCSYAGSYTGVF
GGGTKLTVL
(008-218-HC)
SEQ ID NO: 13
EVQLVESGGGLVQPGRSLRLSCAASGFTFDDYAMHWVRQVPGKGLEWVS
GISWSSGSIGYADSVKGRFTISRDNAKNSLYLQMNSLRAEDTALYYCAK
DRASSWYAYGMDVWGQGTLVTV
(008-218-LC)
SEQ ID NO: 14
NFMLTQPHSVSESPGKTVTISCTRSSGSIASNYVQWYQQRPGSSPTTVI
YDDNQRPSGVPNRFSGSIDSSSNSASLIISGLKTEDEADYYCQSTRVFG
GGTKLTVL

In a specific embodiment subject matter of the present invention is an antibody or antibody fragment or non-Ig scaffold binding to a binding region of an anti-NMDAR1 antibody according to the present invention wherein the binding region of said anti-NMDAR1 antibody is comprised in a sequence that is selected from a group consisting of the following sequences:

| | |
|---|---|
| SEQ ID NO: 15 | GFTFSSYG |
| SEQ ID NO: 16 | IWYDGSNK |
| SEQ ID NO: 17 | ARRHYDFDAFDI |
| SEQ ID NO: 18 | SSDVGGYNY |
| SEQ ID NO: 19 | EVS |
| SEQ ID NO: 20 | SSYTSSSTLYV |
| SEQ ID NO: 21 | GGSISSSNW |
| SEQ ID NO: 22 | IYHSGNT |
| SEQ ID NO: 23 | ARDVSGGVNWFDP |
| SEQ ID NO: 24 | SGSIASNY |
| SEQ ID NO: 25 | EDN |
| SEQ ID NO: 26 | QSYDSSTVV |
| SEQ ID NO: 27 | GYSFTTFW |
| SEQ ID NO: 28 | IYPGDSDT |
| SEQ ID NO: 29 | ARSAVFDY |
| SEQ ID NO: 30 | QSVSSN |
| SEQ ID NO: 31 | GAS |
| SEQ ID NO: 32 | QQYNNWPTSWT |
| SEQ ID NO: 33 | GYSFTSYW |
| SEQ ID NO: 34 | IYPGDSD |
| SEQ ID NO: 35 | ARDYGDYYFDY |
| SEQ ID NO: 36 | SSDVGGYNY |
| SEQ ID NO: 37 | DVS |
| SEQ ID NO: 38 | CSYAGSYTGV |
| SEQ ID NO: 39 | GFTFDDYG |
| SEQ ID NO: 40 | INWSGADT |
| SEQ ID NO: 41 | AREVGIAVTGYWFDP |
| SEQ ID NO: 42 | HSES |
| SEQ ID NO: 43 | DDS |
| SEQ ID NO: 44 | QVWDSSSDHPGVV |
| SEQ ID NO: 45 | GYSFTSYW |
| SEQ ID NO: 46 | IYPGDSDT |
| SEQ ID NO: 47 | ARDYGDYYFDY |
| SEQ ID NO: 48 | SSDVGGYNY |
| SEQ ID NO: 49 | DVS |
| SEQ ID NO: 50 | CSYAGSYTGV |
| SEQ ID NO: 51 | GFTFDDYA |
| SEQ ID NO: 52 | ISWSSGSI |
| SEQ ID NO: 53 | AKDRASSWYAYGMDV |
| SEQ ID NO: 54 | SGSIASNY |
| SEQ ID NO: 55 | DDN |
| SEQ ID NO: 56 | QSTRV |

The binding region for the antibody or antibody fragment or non-Ig scaffold may comprise one or several of the above mentioned sequences. The binding region for the antibody or antibody fragment or non-Ig scaffold may comprise or consist of one or several of the above mentioned sequences. In a specific embodiment subject matter of the present invention is an antibody or antibody fragment or non-Ig scaffold that binds specifically to a binding region of an anti-NMDAR1 antibody as depicted above. Specifically binding in this context means that the antibody or antibody fragment or non-Ig scaffold binds to an anti-NMDAR1 antibody that exhibits at least one of the above described binding regions but does not bind to antibodies that do not exhibit at least one of the above described binding regions.

As the anti-NMDAR1 antibody exhibits a three dimensional structure because of the folding of the antibody, the antibody or antibody fragment or non-Ig scaffold according to the present invention may bind to more than one of the above sequences. Due to the three dimensional structure of proteins the binding region of the antibody or antibody fragment or non-Ig scaffold according to the present invention may consist of non-linear epitopes at least partially overlapping with at least one of the above sequences. Partially overlapping means that at least one, or two, or three, or four, or five amino acids of at least one of the above sequences is bound by the binding region of the antibody or antibody fragment or non-Ig scaffold.

An antibody or antibody fragment or non-Ig scaffold binding to a binding region of an anti-NMDAR1 antibody is synonymously to the term NMDAR1 antibody antibody or NMDAR1 antibody antibody fragment or NMDAR1 antibody non-Ig scaffold, respectively, and is equal to an antibody binding to the NMDAR1 antibody or an antibody fragment binding to the NMDAR1 antibody or a non-Ig scaffold binding to the NMDAR1 antibody and means an anti-NMDAR1 antibody-antibody or an anti-NMDAR1 antibody-antibody fragment or an anti-NMDAR1 antibody-non-Ig scaffold throughout the description and claims.

In one specific embodiment of the invention the antibody binding to the anti-NMDAR1 antibody or antibody fragment binding to the anti-NMDAR1 antibody or non-Ig scaffold binding to the anti-NMDAR1 antibody said antibody or antibody fragment or non-Ig scaffold binds to a region of preferably at least one, or at least two, or at least 3, or preferably at least 4, or at least 5 amino acids within the sequence(s) of the binding region.

In one specific embodiment of the invention the antibody binding to the anti-NMDAR1 antibody or antibody fragment binding to the anti-NMDAR1 antibody or non-Ig scaffold binding to the anti-NMDAR1 antibody said antibody or antibody fragment or non-Ig scaffold binds to a region that overlaps with or contains at least 3, preferably at least 4, preferably at least 5 amino acids comprised within the above sequences SEQ ID Nos: 1 to 56. As above outlined the binding region for the antibody or antibody fragment or non-Ig scaffold may comprise one or several of the above mentioned sequences or parts of one or several of the above mentioned sequences. Such a part comprises at least one, or two, or three, or four, or five amino acids.

In one specific embodiment the term “anti-NMDAR antibody” is understood as “anti-N-methyl-D-aspartate (NMDA) receptor-1 antibody” throughout the entire specification and claims. Accordingly, in said specific embodiment term “NMDAR” is understood as “N-methyl-D-aspartate (NMDA) receptor-1”. Accordingly, in said specific embodiment the term “the antibody or antibody fragment or non-Ig scaffold binding to the anti-NMDAR antibody” is understood as antibody or antibody fragment or non-Ig scaffold binding to the N-methyl-D-aspartate (NMDA) receptor-1. The person skilled in the art understands that N-methyl-D-aspartate (NMDA) receptor-1 is the NR1 sub-unit of the NMDA receptor.

An antibody or fragment according to the present invention is a protein including one or more polypeptides substantially encoded by immunoglobulin genes that specifically binds an antigen. The recognized immunoglobulin genes include the kappa, lambda, alpha (IgA), gamma (IgG₁, IgG₂, IgG₃, IgG₄), delta (IgD), epsilon (IgE) and mu (IgM) constant region genes, as well as the myriad immunoglobulin variable region genes. Full-length immunoglobulin light chains are generally about 25 kDa or 214 amino acids in length. Full-length immunoglobulin heavy chains are generally about 50 kDa or 446 amino acid in length. Light chains are encoded by a variable region gene at the NH2-terminus (about 110 amino acids in length) and a kappa or lambda constant region gene at the COOH-terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)₂, as well as bifunctional hybrid antibodies and single chains. An immunoglobulin light or heavy chain variable region includes a framework region interrupted by three hypervariable regions, also called complementarity determining regions (CDRs) (see E. Kabat et al., U.S. Department of Health and Human Services, 1983). As noted above, the CDRs are primarily responsible for binding to an epitope of an antigen. An immune complex is an antibody, such as a monoclonal antibody, chimeric antibody, humanized antibody or human antibody, or functional antibody fragment, specifically bound to the antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody, although other mammalian species can be used, or the variable region can be produced by molecular techniques. Methods of making chimeric antibodies are well known in the art, e.g., see U.S. Pat. No. 5,807,715. A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Exemplary conservative substitutions are those such as gly, ala; val, ile, leu; asp, glu; asn, gln; ser, thr; lys, arg; and phe, tyr. Humanized immunoglobulins can be constructed by means of genetic engineering (e.g., see U.S. Pat. No. 5,585,089). A human antibody is an antibody wherein the light and heavy chain genes are of human origin. Human antibodies can be generated using methods known in the art. Human antibodies can be produced by immortalizing a human B cell secreting the antibody of interest. Immortalization can be accomplished, for example, by EBV infection or by fusing a human B cell with a myeloma or hybridoma cell to produce a trioma cell. Human antibodies can also be produced by phage display methods (WO 91/17271; WO 92/001047), or selected from a human combinatorial monoclonal antibody library (see the Morphosys website). Human antibodies can also be prepared by using transgenic animals carrying a human immunoglobulin gene (for example, see WO 93/12227; and WO 91/10741).

Thus, the antibody according to the present invention may have the formats known in the art. Examples are human antibodies, monoclonal antibodies, humanized antibodies, chimeric antibodies, CDR-grafted antibodies. In a preferred embodiment antibodies according to the present invention are recombinantly produced antibodies as e.g. IgG, a typical full-length immunoglobulin, or antibody fragments containing at least the variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH3 domain; bivalent Fab or multivalent Fab, e.g. formed via multimerization with the aid of a heterologous domain, e.g. via dimerization of dHLX domains, e.g. Fab-dHLX-FSx2; F(ab′)₂-fragments, scFv-fragments, multimerized multivalent or/and multispecific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulines and numerous others.

In a preferred embodiment the antibody format is selected from the group comprising Fv fragment, scFv fragment, Fab fragment, scFab fragment, F(ab)₂ fragment and scFv-Fc Fusion protein. In another preferred embodiment the antibody format is selected from the group comprising scFab fragment, Fab fragment, scFv fragment and bioavailability optimized conjugates thereof, such as PEGylated fragments. One of the most preferred formats is the scFab format. For illustration of antibody formats please see FIGS. 1a, 1b and 1c.

In addition to antibodies other biopolymer scaffolds are well known in the art to complex a target molecule and have been used for the generation of highly target specific biopolymers. Examples are aptamers, spiegelmers, anticalins and conotoxins.

Subject matter of the present invention is an antibody or antibody fragment or non-Ig scaffold binding to a binding region of an anti-NMDAR1 antibody according to the present invention wherein Non-Ig scaffolds may be protein scaffolds and may be used as antibody mimics as they are capable to bind to ligands or antigens. Non-Ig scaffolds may be selected from the group comprising tetranectin-based non-Ig scaffolds (e.g. described in US 2010/0028995), fibronectin scaffolds (e.g. described in EP 1 266 025); lipocalin-based scaffolds (e.g. described in WO 2011/154420); ubiquitin scaffolds (e.g. described in WO 2011/073214), transferrin scaffolds (e.g. described in US 2004/0023334), protein A scaffolds (e.g. described in EP 2 231 860), ankyrin repeat based scaffolds (e.g. described in WO 2010/060748), microproteins preferably microproteins forming a cysteine knot) scaffolds (e.g. described in EP 2 314 308), Fyn SH3 domain based scaffolds (e.g. described in WO 2011/023685) EGFR-A-domain based scaffolds (e.g. described in WO 2005/040229) and Kunitz domain based scaffolds (e.g. described in EP 1 941 867), and Avimers (US 20050053973 A1), and CTLA4-based scaffolds (WO 00/60070), and Armadillo repeat proteins (US 20110224100 A1). Subject matter of the present invention is an antibody or antibody fragment or non-Ig scaffold binding to a binding region of an anti-NMDAR1 subunit antibody according to the present invention wherein non-Ig scaffolds may be non-protein scaffolds and may be used as antibody mimics as they are capable to bind to ligands or antigens. Such non-Ig scaffolds may be selected from the group comprising oligonucleotide aptamers: RNA aptamers, DNA aptamers and L-RNA-aptamers (Spiegelmers).

In a specific embodiment of the present invention the subject matter of the present invention is a non-IgG scaffold. In a more specific embodiment said non-IgG scaffold is a non-peptid and non-protein non-IgG scaffold. In a more specific embodiment said non-IgG scaffold is an oligonucleotide aptamer selected from the group comprising RNA aptamers, DNA aptamers and L-RNA-aptamers (Spiegelmers).

To generate aptamers using the SELEX system (U.S. Pat. No. 5,270,163), the target structure (human monoclonal NMDAR1 antibodies) is incubated with a library of >10¹⁵ random aptamers containing almost any three-dimensional structure. Several rounds of selection and amplification result in highly specific and affine aptamers (FIG. 2). The robot-assisted selection process is highly standardized and contains the following steps: aptamer library and antibody incubation, amplification and purification of bound aptamers, repeat approximately 9 SELEX rounds, next generation sequencing of aptamers during the selection process, affinity control using plasmon resonance.

Subject matter of the present invention is an antibody or antibody fragment or non-Ig scaffold binding to a binding region of an anti-NMDAR1 antibody according to the present invention wherein said antibody or antibody fragment or non-Ig scaffold exhibits an affinity towards said binding region of an anti-NMDAR1 antibody in such that the dissociation constant (K_{D}) is lower than 10⁻⁷ M, preferred 10⁻⁸ M, preferred K_{D} is lower than 10⁻⁹ M, most preferred lower than 10⁻¹⁰ M to said binding region of the anti-NMDAR1 antibody. The binding affinity may be determined in an assay according to Example 4. Example 4 describes a surface plasmon resonance analysis (Biacore).

Subject matter of the present invention is an antibody or antibody fragment or non-Ig scaffold binding to a binding region of an anti-NMDAR1 antibody according to the present invention wherein said antibody or antibody fragment or non-Ig scaffold binds specifically to said anti-NMDAR1 antibody.

An antibody or antibody fragment or non-Ig scaffold binding to a binding region of an anti-NMDAR1 antibody according to the present invention captures anti-NMDAR1-antibodies in vivo or alternatively captures anti-NMDAR1-antibodies ex-vivo in solution and/or removes and/or depletes anti-NMDAR1-antibodies from bodily fluids. The term “captures anti-NMDAR1-antibodies in vivo” may be understood as inhibits and/or blocks and/or hinders and/or abolishes and/or suppresses NMDAR1 binding by anti-NMDAR1-antibodies or neutralizes and/or scavenges and/or intercepts anti-NMDAR1-antibodies. In particular, an antibody or antibody fragment or non-Ig scaffold binding to a binding region of an anti-NMDAR1 antibody according to the present invention may capture and/or remove anti-NMDAR1-autoantibodies from bodily fluid while not capturing and/or removing >80% of non-NMDAR1 specific Ig-molecules, in particular >90% of non-NMDAR1 specific Ig-molecules, in particular >99% of non-NMDAR1 specific Ig-molecules. The percentages relate to photometrical measurement at 450 nm as exemplified below:

Binding and/or removal of Ig-molecules may be determined using an ELISA comparing patient samples taken before and after treatment with an antibody or antibody fragment or non-Ig scaffold binding to a binding region of an anti-NMDAR1 antibody. The assay employs an antibody specific for Human Ig coated on a 96-well plate. Samples are pipetted into the wells and Ig present in a sample is bound to the wells by the immobilized antibody. The wells are washed and biotinylated anti-Human Ig antibody is added. After washing away unbound biotinylated antibody, HRP-conjugated streptavidin is pipetted to the wells. The wells are again washed, a TMB substrate solution is added to the wells and color (measured photometrically at 450 nm) develops in proportion to the amount of Ig bound.

In particular, an antibody or antibody fragment or non-Ig scaffold binding to a binding region of an anti-NMDAR1 antibody according to the present invention binds to and/or removes anti-NMDAR1-autoantibodies from bodily fluids and does not bind or does not essentially bind to other Ig-molecules specific for pathogens, tumor antigens or known to have protective physiological functions.

A method for the ex vivo selective depletion of the anti-AChR autoantibodies from patients' plasma through the construction of “immunoadsorbent” columns carrying AChR domains has been described in Tzartos et al. 2008. Ann N Y Acad Sci. 1132:291-9. The same method may be used according to the present invention for selective depletion of anti-NMDAR1 antibody from patient plasma. A patient may be a human or animal subject.

In a specific embodiment said bodily fluid maybe selected from the group consisting of serum, plasma, cerebrospinal fluid (CSF) and full blood, urine, saliva and amniotic fluid.

Subject matter of the present invention is an antibody or antibody fragment or non-Ig scaffold binding to a binding region of an anti-NMDAR1 antibody according to the present invention wherein said antibody or antibody fragment or non-Ig scaffold neutralizes the anti-NMDAR1 antibodies or neutralizes the biological effect of said anti-NMDAR1 antibodies.

In one embodiment a neutralizing antibody or antibody fragment or non-Ig scaffold binding to a binding region of an anti-NMDAR1 antibody according to the present invention inhibits the binding of purified anti-NMDAR1-antibodies or patient autoantibodies in bodily fluids to the recombinantly expressed NMDAR1 in vitro. The inhibition of binding may be determined in an inhibition cell and tissue assay according to Example 5.1 and/or Example 5.2:
Aptamer inhibits binding of purified anti-NMDAR1-antibody to transfected HEK293-cells expressing the NMDAR1 (GRIN1) (see e.g. Example as in WO 2012076000 A2, example 1; Euroimmune: Autoimmune-Enzephalitis Mosaik 1);
Aptamer inhibits binding of anti-NMDAR1-antibody from blood/serum/CSF of patients to transfected HEK293-cells expressing the NMDAR1 (GRIN1) (Example as in WO 2012076000 A2)

In one embodiment a neutralizing antibody or antibody fragment or non-Ig scaffold binding to a binding region of an anti-NMDAR1 antibody according to the present invention inhibits the binding of purified anti-NMDAR1-antibodies to NMDAR1-expressing tissues according to Example 5.2:
Immunohistochemistry: Aptamer inhibits binding of anti-NMDAR1-antibody to tissues expressing the NMDAR1.

In one embodiment a neutralizing antibody or antibody fragment or non-Ig scaffold binding to a binding region of an anti-NMDAR1 antibody according to the present invention inhibits the anti-NMDAR1-antibody-mediated down regulation of NMDAR-positive synaptic clusters as shown in Example 5.4, FIG. 7:
Aptamer inhibits anti-NMDAR1-autoantibody-mediated downregulation.

Subject matter of the present invention is an antibody or antibody fragment or non-Ig scaffold binding to a binding region of an anti-NMDAR1 antibody according to the present invention wherein said antibody or antibody fragment or non-Ig scaffold is a monospecific antibody or antibody fragment or non-Ig scaffold.

Monospecific antibody or monospecific antibody fragment or monospecific non-Ig scaffold means that said antibody or antibody fragment or non-Ig scaffold binds to one specific region of preferably at least 1, preferably at least 2, preferably at least 3, preferably at least 4, or at least 5 amino acids within the sequence(s) of the binding region. In one specific embodiment of the invention said mono specific antibody or mono specific antibody fragment or monospecific non-Ig scaffold binds to a region that overlaps with or contains at least 1, preferably at least 2, preferably at least 3, preferably at least 4, preferably at least 5 amino acids comprised within the above sequences with SEQ ID Nos: 1 to 56. As above outlined the binding region for the antibody or antibody fragment or non-Ig scaffold may comprise one or several of the above mentioned sequences.

Monospecific antibody or monospecific antibody fragment or monospecific non-Ig scaffold are antibodies or antibody fragments or non-Ig scaffolds that all have affinity for the same antigen.

Monospecific antibodies or fragments or non-Ig scaffolds according to the invention are antibodies or fragments or non-Ig scaffolds that all have affinity for the same antigen. Monoclonal antibodies are monospecific, but monospecific antibodies may also be produced by other means than producing them from a common germ cell.

Antibodies may be produced by means of active immunization according to the following procedure:

Synthetically produced peptide sequences (according to the above given NMDAR1-binding sequences of the monoclonal recombinant NMDAR1 antibodies) or the monoclonal human NMDAR1 antibodies (after cleavage of the Fc part) are used for active immunization. 20 μg protein per mouse is emulsified with Complete Freund's Adjuvant (CFA) and 200 μl emulsion injected subcutaneously. Repeated booster immunizations are performed with 20 μg protein per mouse in Incomplete Freund's Adjuvant (IFA) after 4 and 8 weeks via intraperitoneal injection. Antibody-producing B cells are harvested from spleens, screened for cell clones that react with the desired epitope with sufficient affinity, and isolated for monoclonal antibody generation following standard protocols including yeast surface display in combination with high-throughput fluorescence-activated cell sorting (e.g. Doerner et al. 2014. FEBS Lett. 21; 588(2):278-87).

Humanization of murine antibodies may be conducted according to the following procedure: For humanization of an antibody of murine origin the antibody sequence is analyzed for the structural interaction of framework regions (FR) with the complementary determining regions (CDR) and the antigen. Based on structural modeling an appropriate FR of human origin is selected and the murine CDR sequences are transplanted into the human FR. Variations in the amino acid sequence of the CDRs or FRs may be introduced to regain structural interactions, which were abolished by the species switch for the FR sequences. This recovery of structural interactions may be achieved by random approach using phage display libraries or via directed approach guided by molecular modeling. (Almagro et al. 2008. Front Biosci.13:1619-33.)

In a preferred embodiment the antibody format of the present invention is selected from the group comprising Fv fragment, scFv fragment, Fab fragment, scFab fragment, F(ab)₂ fragment and scFv-Fc Fusion protein. In another preferred embodiment the antibody format is selected from the group comprising scFab fragment, Fab fragment, scFv fragment and bioavailability optimized conjugates thereof, such as PEGylated fragments. One of the most preferred formats is scFab format.

In another embodiment, the antibody, antibody fragment, or non-Ig scaffold is a full length antibody, antibody fragment, or non-Ig scaffold.

In a more preferred embodiment the antibody or antibody fragment or non-Ig scaffold is directed to and can bind to an epitope of at least 1, preferably at least 2, preferably at least 3 or 4 or 5 amino acids in length contained in the binding region.

The term NMDAR1 antibody antibody or NMDAR1 antibody antibody fragment or

NMDAR1 antibody non-Ig scaffold is equal to an antibody binding to the NMDAR1 antibody or an antibody fragment binding to the NMDAR1 antibody or a non-Ig scaffold binding to the NMDAR1 antibody and means an anti-NMDAR1 antibody-antibody or an anti-NMDAR1 antibody-antibody fragment or an anti-NMDAR1 antibody-non-Ig scaffold throughout the description and claims.

Subject matter of the present invention is an antibody or antibody fragment or non-Ig scaffold binding to a binding region of an anti-NMDAR1 subunit antibody according to the present invention for use in therapy of a disease or a condition in a subject said disease or condition being associated with anti-NMDAR1 subunit antibodies and in a specific embodiment having in addition at least one clinical symptom or clinical condition selected from the group comprising the clinical symptoms/conditions according to the following list (ICD numbers in parentheses refer to the WHO International Classification of Diseases which defines the clinical conditions):
psychiatric abnormalities including depression (F32), mania with psychotic symptoms (F30.2), anxiety (F06.4), phobic anxiety (F40), delusions (F22.0), obsessive-compulsive disorder (F42), organic delusional disorder (F06.3), catatonia (F06.1, F20.2), acute polymorphic psychotic disorder (F23.0, F23.1), dissociative disorders (F44)
movement disorders including dyskinesias/dystonia (G24), myoclonus (G25.3), tremor (G25.0, G25-1, G25-2), tics (F95, G25.69)
epileptic seizures (G40)
hypoventilation (R06.89)
mild cognitive impairment (F06.7)
dementia in Alzheimer's disease (F00), vascular dementia (F01), dementia in other diseases (F02)
pregnancy.

Subject matter of the present invention is an antibody or antibody fragment or non-Ig scaffold binding to a binding region of an anti-NMDAR1 subunit antibody according to the present invention for use in therapy of a disease or a condition in a subject said disease or condition being associated with anti-NMDAR1 subunit antibodies as defined above wherein the therapeutic effect is based on the binding and/or blocking and/or removal of said NMDAR1 antibody according to the invention from bodily fluid of said patients.

In contrast to treatments of autoimmune disorders according to the prior art therapies, e.g. treatment with intravenous immunoglobulins (IVIG) or unselective plasma exchange the therapy of the present invention is based on the specific effect against said NMDAR1 antibodies. It is well known and thus forms the basis for routine guidelines in Neurology that the clinical improvement in NMDAR antibody-associated autoimmune encephalitis is strongly associated with the decline of antibody titers following therapy (Gresa-Arribas et al. 2014, Lancet Neurol 13(2):167-77; Dogan Onugoren et al. 2016, Neurol Neuroimmunol Neuroinflamm. 26; 3(2):e207).

The beneficial effect of IVIG in autoimmune disorders is not by binding and/or neutralizing and/or blocking pathogenic antibodies via anti-idiotype antibodies. Rather, among many open questions regarding the mechanisms of IVIG therapy, it is well accepted that IVIG act mainly by negative feedback on antibody-producing cells via the inhibitory Fc-gamma receptor Fc γ IIB, by modulation of T cell activation, regulation of peripheral tolerance and release of chemoattractants via the Fc part (Nimmerjahn & Ravetch 2008, Nature Review Immunology 8(1):34-47). Data from human clinical trials demonstrated that the Fc fragment contains most of the anti-inflammatory activity (Schwab, Lux, Nimmerjahn 2015, Cell Rep 20; 13(3):610-20). Along this pathway, IVIG down-regulate not only the disease specific pathogenic antibodies, but also (and this is unwanted and can be overcome with the approach according to the present invention) all beneficial antibodies in the human body. Thus, the therapy of the present inventions provides a therapy with less side effects but having the same or better efficacy when compared to the prior art methods.

The surprising finding of the present invention is that the antibody or antibody fragment or non-Ig scaffold binding to a binding region of an anti-NMDAR1 subunit antibody according to the present invention may bind to the majority of autoantibodies in patients with said disease or condition being associated with anti-NMDAR1 subunit antibodies and/or having in addition at least one clinical symptom or clinical condition as above described. Indeed the binding regions of the autoantibodies isolated from different patients exhibit a surprising degree of similarity. For instance the CDR2 of the light chain of all isolated autoantibodies consists of only three amino acids and is dominated in 5/6 sequences by acidic amino acids (D/E). Further, the CDR 1 and also CDR3 of the heavy chain show also surprising analogies:

| | CDR 1 | CDR 2 | CDR 3 |
|---|---|---|---|
| 003-109-HC | GFTFSSYG | IWYDGSNK | ARRHYDFDAFDI |
| 003-102-HC | GGSISSSNW | IYHSGNT | ARDVSGGVNWFDP |
| 007-168-HC | GYSFTTFW | IYPGDSDT | ARSAVFDY |
| 007-169-HC | GYSFTSYW | IYPGDSD | ARDYGDYYFDY |
| 007-124-HC | GFTFDDYG | INWSGADT | AREVGIAVTGYWFDP |
| 008-218-HC | GFTFDDYA | ISWSSGSI | AKDRASSWYAYGMDV |
| 003-109-LC | SSDVGGYNY | EVS | SSYTSSSTLYV |
| 003-102-LC | SGSIASNY | EDN | QSYDSSTVV |
| 007-168-LC | QSVSSN | GAS | QQYNNWPTSWT |
| 007-169-LC | SSDVGGYNY | DVS | CSYAGSYTGV |
| 007-124-LC | HSES | DDS | QVWDSSSDHPGVV |
| 008-218-LC | SGSIASNY | DDN | QSTRV |

anti-NMDAR1 subunit autoantibodies from many different patients can be still bound by the same antibody or antibody fragment or non-Ig scaffold binding to a binding region of an antibody according to the present invention. This is based on the experimental finding that monoclonal antibodies from different patients all bind to a very small epitope on the aminoterminal domain of the NMDA receptor. In fact, mutation of only one amino acid is resulting in complete loss of antibody binding to the NMDA receptor, and this amino acid change (N to Q) is expected to result in a very local structural change rather than in a change of the three-dimensional structure of the receptor. Thus, one or a relatively small pool of antibodies or antibody fragments or non-Ig scaffolds binding to a binding region of an anti-NMDAR1 subunit antibody according to the present invention are potentially able to block auto-antibodies from different patients. Another line of evidence is the fact that we could identify unmutated human antibodies against the NMDAR (Kreye et al. 2016, Brain). These antibodies comprise the so-called germ-line configuration (also called ‘naturally occurring antibodies’), i.e. they are continuously generated by the body, not only in patients but in everyone and will thus stochastically be present also in previously healthy persons. These naturally occurring antibodies are thought to mainly positively participate in homeostasis, removal of dead cells, but can—in the case of NMDAR antibodies—also be detrimental to nerve cells as shown recently (Kreye et al. 2016, Brain). Due to the sequence code of naturally occurring NMDAR antibodies in the normal (healthy) genetic repertoire, the data suggest that there is an evolutionary restriction to a limited number of sequences. This is in perfect agreement with the abovementioned fact that all monoclonal NMDAR antibodies identified so far from different patients rely on such a small epitope in the aminoterminal domain of the receptor.

In a specific embodiment the patient or subject having a disease or a condition being associated with anti-NMDAR1 subunit antibodies and in a specific embodiment having in addition at least one clinical symptom or clinical condition as above described is stratified for having an anti-NMDAR1 antibody with a binding region in a bodily fluid wherein the binding region of said anti-NMDAR1 antibody is comprised in or consists of a sequence that is selected from a group consisting of the following sequences:

| | |
|---|---|
| SEQ ID NO: 15 | GFTFSSYG |
| SEQ ID NO: 16 | IWYDGSNK |
| SEQ ID NO: 17 | ARRHYDFDAFDI |
| SEQ ID NO: 18 | SSDVGGYNY |
| SEQ ID NO: 19 | EVS |
| SEQ ID NO: 20 | SSYTSSSTLYV |
| SEQ ID NO: 21 | GGSISSSNW |
| SEQ ID NO: 22 | IYHSGNT |
| SEQ ID NO: 23 | ARDVSGGVNWFDP |
| SEQ ID NO: 24 | SGSIASNY |
| SEQ ID NO: 25 | EDN |
| SEQ ID NO: 26 | QSYDSSTVV |
| SEQ ID NO: 27 | GYSFTTFW |
| SEQ ID NO: 28 | IYPGDSDT |
| SEQ ID NO: 29 | ARSAVFDY |
| SEQ ID NO: 30 | QSVSSN |
| SEQ ID NO: 31 | GAS |
| SEQ ID NO: 32 | QQYNNWPTSWT |
| SEQ ID NO: 33 | GYSFTSYW |
| SEQ ID NO: 34 | IYPGDSD |
| SEQ ID NO: 35 | ARDYGDYYFDY |
| SEQ ID NO: 36 | SSDVGGYNY |
| SEQ ID NO: 37 | DVS |
| SEQ ID NO: 38 | CSYAGSYTGV |
| SEQ ID NO: 39 | GFTFDDYG |
| SEQ ID NO: 40 | INWSGADT |
| SEQ ID NO: 41 | AREVGIAVTGYWFDP |
| SEQ ID NO: 42 | HSES |
| SEQ ID NO: 43 | DDS |
| SEQ ID NO: 44 | QVWDSSSDHPGVV |
| SEQ ID NO: 45 | GYSFTSYW |
| SEQ ID NO: 46 | IYPGDSDT |
| SEQ ID NO: 47 | ARDYGDYYFDY |
| SEQ ID NO: 48 | SSDVGGYNY |
| SEQ ID NO: 49 | DVS |
| SEQ ID NO: 50 | CSYAGSYTGV |
| SEQ ID NO: 51 | GFTFDDYA |
| SEQ ID NO: 52 | ISWSSGSI |
| SEQ ID NO: 53 | AKDRASSWYAYGMDV |
| SEQ ID NO: 54 | SGSIASNY |
| SEQ ID NO: 55 | DDN |
| SEQ ID NO: 56 | QSTRV |

Thus, the patient in need of a therapy with the antibody or antibody fragment or non-Ig scaffold binding to a binding region of an anti-NMDAR1 subunit antibody according to the present invention may be selected by determining the presence of an anti-NMDR1 antibody as above defined in a sample of bodily fluid of a subject in order to determine whether said subject is in need of such therapy wherein said subject has a disease or condition being associated with anti-NMDAR1 antibodies as defined above.

One typical example would be the anti-NMDAR encephalitis, a severe encephalitis predominantly affecting young females, but also children and men of all ages (Dalmau et al. 2008. Lancet Neurol. 7(12):1091-8). High-titer NMDAR1 antibodies are a hallmark of the disorder. Symptoms typically include several of the above list (such as psychiatric abnormalities, movement disorders, epileptic seizures, hypoventilation and the need for intensive care unit treatment), but forms with isolated seizures, cognitive impairment or psychosis can occur.

The above-identified subjects may be in need of a therapy wherein said antibody or antibody fragment or non-Ig scaffold according to the present invention is administered to said subject. Said subject may be a human or animal subject throughout the entire specification.

Thus, a subject that may be in need of a therapy according to the present invention is a subject that has NMDAR1 antibody in a bodily fluid. In another embodiment, a subject that may be in need of a therapy according to the present invention is a subject that has NMDAR1 antibody in a bodily fluid and having in addition at least one clinical symptom or clinical condition selected from the group comprising the clinical symptoms/conditions according to the following list (ICD numbers in parentheses refer to the WHO International Classification of Diseases which defines the clinical conditions):
psychiatric abnormalities including depression (F32), mania with psychotic symptoms (F30.2), anxiety (F06.4), phobic anxiety (F40), delusions (F22.0), obsessive-compulsive disorder (F42), organic delusional disorder (F06.3), catatonia (F06.1, F20.2), acute polymorphic psychotic disorder (F23.0, F23.1), dissociative disorders (F44)
movement disorders including dyskinesias/dystonia (G24), myoclonus (G25.3), tremor (G25.0, G25-1, G25-2), tics (F95, G25.69)
epileptic seizures (G40)
hypoventilation (R06.89)
mild cognitive impairment (F06.7)
dementia in Alzheimer's disease (F00), vascular dementia (F01), dementia in other diseases (F02)
pregnancy.

Subject matter of the present invention is an antibody or antibody fragment or non-Ig scaffold binding to a binding region of an anti-NMDAR1 antibody according to the present invention for use in therapy of a disease or condition in a subject associated with anti-NMDAR1 antibodies according to the present invention wherein said antibody or antibody fragment or non-Ig scaffold is administered in vivo to said subject being in need of such a therapy.

Subject matter of the present invention is an antibody or antibody fragment or non-Ig scaffold binding to a binding region of an anti-NMDAR1 antibody according to the present invention for use in therapy of a disease or condition in a subject associated with anti-NMDAR1 antibodies according to the present invention wherein said antibody or antibody fragment or non-Ig scaffold is administered intravenously or directly into the CSF to said subject being in need of such a therapy.

Subjects in need of said therapy may be treated by ex vivo therapies in another embodiment of the invention.

Thus, subject matter of the present invention is an antibody or antibody fragment or non-Ig scaffold binding to a binding region of an anti-NMDAR1 antibody according to the present invention for use in therapy of a disease or a condition in a subject being in need of such a therapy said disease being associated with anti-NMDAR1 antibodies wherein said antibody or antibody fragment or non-Ig scaffold is used in an ex vivo therapy of said patient. Said patient may be a human or animal subject.

Thus, subject matter of the present invention is an antibody or antibody fragment or non-Ig scaffold binding to a binding region of an anti-NMDAR1 antibody according to the present invention for use in therapy of a disease or a condition in a subject being in need of such a therapy said disease or condition being associated with anti-NMDAR1 antibodies wherein said subject exhibits the presence of anti-NMDAR1 antibodies when measured according to a method as described below. Specifically the presence of anti-NMDAR1 antibodies may be determined with an assay according to Example 1.2

Subject of the present invention is further a pharmaceutical formulation comprising an antibody or fragment or scaffold according to the present invention. Said pharmaceutical formulation may comprise one or more antibody or fragment or scaffold according to the present invention.

Subject of the present invention is further a pharmaceutical formulation comprising an antibody or fragment or non-IgG scaffold according to the present invention wherein said pharmaceutical formulation is a solution, preferably a ready-to-use solution.

Said pharmaceutical formulation may be administered intra-vascular. Said pharmaceutical formulation may be administered via infusion.

In another embodiment subject of the present invention is further a pharmaceutical formulation according to the present invention wherein said pharmaceutical formulation is in a dried state or freeze-dried to be reconstituted before use.

It should be emphasized that the pharmaceutical formulation in accordance with the invention may be administered systemically to a patient, preferably via infusion or intra-vascular. A patient may be a human or animal subject throughout the specification.

Subject matter of the present invention is an antibody or antibody fragment or non-Ig scaffold binding to a binding region of an anti-NMDAR1 antibody according to the present invention for use in therapy of a disease in a patient said disease being associated with anti-NMDAR1 antibodies wherein said antibody or antibody fragment or non-Ig scaffold is to be used in combination with another agent, e.g. either a chemotherapeutic agent or a immunosuppressive agent. Said agent may be selected from the group comprising azathioprine, cyclophosphamide, rituximab, methotrexate, bortezomib, corticosteroids, and mycophenolat mofetil.

Subject matter of the present invention is an antibody or antibody fragment or non-Ig scaffold-coated device for plasma exchange (plasmapheresis) or CSF exchange (liquorpheresis) wherein said antibody or antibody fragment or non-Ig scaffold is an antibody or antibody fragment or non-Ig scaffold according to the present invention.

Methods and extracorporeal systems for apheresis (i.e., the process of withdrawing blood from an individual, removing components from the blood, and returning the blood, or blood depleted of one or more components, to the individual) are known in the art (see, for example, U.S. Pat. Nos. 4,708,713; 5,258,503; 5,386,734; 6,409,696; and Hendrickson et al. 2015. J Clin Apher. doi: 10.1002/jca.21407. [Epub ahead of print]).

Subject matter of the present invention is an antibody or antibody fragment or non-Ig scaffold-coated device for plasma exchange or CSF exchange (liquorpheresis) according to the present invention wherein said antibody or antibody fragment or non-Ig scaffold-coated device is an antibody or antibody fragment or non-Ig scaffold-coated column. Such devices are described e.g. Fresenius Medical Care; “Protein-A-Adsorber Immunosorba®” or for IgE-specific aphereses EP 2696895 A1.

Subject matter of the present invention is the use of an antibody or antibody fragment or non-Ig scaffold according to the present invention for determining the presence of anti-NMDAR1 antibodies in a sample of a bodily fluid of a patient having a disease being associated with anti-NMDAR1 antibodies or in a sample of a bodily fluid in female pregnant subject.

Subject matter of the present invention is a method of determining the presence of an anti-NMDAR1 antibody in a sample of bodily fluid of a subject in order to determine whether said subject is in need of a therapy according to the present invention wherein said subject has a disease or condition being associated with anti-NMDAR1 antibodies and the method comprising
contacting a sample of bodily fluid obtained from said subject with at least one antibody or antibody fragment or non-Ig scaffold according to the present invention
determining the presence of said anti-NMDAR1 antibodies in said sample
wherein in case an anti-NMDAR1 antibody is present in said sample said subject may have a disease or condition associated with anti-NMDAR1 antibodies and may be in need of a therapy according to the present invention.

Subject matter of the present invention is a method of determining the presence of an anti-NMDAR1 antibody in a sample of bodily fluid of a subject in order to determine whether said subject is in need of a therapy according to the present invention wherein said subject has a disease or condition being associated with anti-NMDAR1 antibodies and the method comprising
contacting a sample of bodily fluid obtained from said subject with at least two antibodies or antibody fragments or non-Ig scaffolds according to the present invention
determining the presence of said anti-NMDAR1 antibodies in said sample
wherein in case an anti-NMDAR1 antibody is present in said sample said subject may have a disease or condition associated with anti-NMDAR1 antibodies and may be in need of a therapy according to the present invention.

In particular the NMDAR1 antibody in a sample of bodily fluid is characterized by having a binding region that is comprised in one ore more sequences wherein said one or more sequence is selected from a group consisting of the following sequences:

(003-109-HC)
SEQ ID NO: 1
VQLVESGGGVVQPGRSLRLSCAASGFTFSSYGMHWVRQAPGKGLEWVAV
IWYDGSNKYYADSVKGRFTISRDNSKNTLYLQMNSLRAEDTAVYYCARR
HYDFDAFDIWGQGTMVTVSS
(003-109-LC)
SEQ ID NO: 2
QSALTQPASVSGSPGQSITISCTGTSSDVGGYNYVSWYQQHPGKAPKLM
IYEVSNRPSGVSNRFSGSKSGNTASLTISGLQAEDEADYYCSSYTSSST
LYVFGTGTKVTVL
(003-102-HC)
SEQ ID NO: 3
QVQLQESGPGLVKPSGTLSLTCAVSGGSISSSNWWSWVRQPPGKGLEWI
GEIYHSGNTNYNPSLKSRVTVSVDKSKNQFSLKLTSVTAADTAVYYCAR
DVSGGVNWFDPWGQGTLVTVSS
(003-102-LC)
SEQ ID NO: 4
NFMLTQPHSVSESPGKTVTISCTRSSGSIASNYVQWYQQRPGSAPTTVI
YEDNQRPSGVPDRFSGSIDSSSNSASLTISGLKTEDEADYYCQSYDSST
VVFGGGTKLTVL
(007-168-HC)
SEQ ID NO: 5
VQLVQSGAEAKKPGESLKISCKASGYSFTTFWIGWVRQMPGSGLEWIGI
IYPGDSDTRYSPSFQGHVTISADRSTSTAYLQWSSLKASDTAMYYCARS
AVFDYWGQGTLVTVSS
(007-168-LC)
SEQ ID NO: 6
EIVMTQSPATLSVSPGGRATLSCRASQSVSSNLAWYQQKPGQAPRLLIY
GASTRATGIPVRFSGSGSGTEFTLTISSLQSEDFAVYYCQQYNNWPTSW
TFGQGTKVEIK
(007-169-HC)
SEQ ID NO: 7
EVQLVQSGAEVKKPGESLKISCKGSGYSFTSYWIGWVRQMPGKGLEWMG
IIYPGDSDTRYSPSFQGQVTISADKSISTAYLQWSSLKASDTAMYYCAR
DYGDYYFDYWGQGTLVTVSS
(007-169-LC)
SEQ ID NO: 8
QSALTQPRSVSGSPGQSVTISCTGTSSDVGGYNYVSWYQQHPGKAPKLM
IYDVSKRPSGVPDRFSGSKSGNTASLTISGLQAEDEADYYCCSYAGSYT
GVFGEGTKLTVL
(007-124-HC)
SEQ ID NO: 9
EVQLVESGGGVGRPGGSLRLSCAASGFTFDDYGMSWVRQVPGKGLEWVS
GINWSGADTGYADSVKGRFTISRDNAKNSLYLQMNSLRVEDTALYHCAR
EVGIAVTGYWFDPWGQGTLVTV
(007-124-LC)
SEQ ID NO: 10
SYELTQPPSVSVAPGQTARISCGGNHSESVHWYQQKPGQAPVLVVYDDS
DRPSGIPERFSGSKSGNTATLTISRVEGGDEAEYYCQVWDSSSDHPGVV
FGGGTKLTVL
(007-142-HC)
SEQ ID NO: 11
EVQLVQSGAEVKKPGESLKISCKGSGYSFTSYWIGWVRQMPGKGLEWMG
IIYPGDSDTRYSPSFQGQVTISADKSISTAYLQWSSLKASDTAMYYCAR
DYGDYYFDYWGQGTLVTVSS
(007-142-LC)
SEQ ID NO: 12
LTQPRSVSGSPGQSVTISCTGTSSDVGGYNYVSWYQQHPGKAPKLMIYD
VSKRPSGVPDRFSGSKSGNTASLTISGLQAEDEADYYCCSYAGSYTGVF
GGGTKLTVL
(008-218-HC)
SEQ ID NO: 13
EVQLVESGGGLVQPGRSLRLSCAASGFTFDDYAMHWVRQVPGKGLEWVS
GISWSSGSIGYADSVKGRFTISRDNAKNSLYLQMNSLRAEDTALYYCAK
DRASSWYAYGMDVWGQGTLVTV
(008-218-LC)
SEQ ID NO: 14
NFMLTQPHSVSESPGKTVTISCTRSSGSIASNYVQWYQQRPGSSPTTVI
YDDNQRPSGVPNRFSGSIDSSSNSASLIISGLKTEDEADYYCQSTRVFG
GGTKLTVL

And wherein said binding region for the antibody or antibody fragment or non-Ig scaffold comprises or consists of one or several of the below mentioned sequences:

| | |
|---|---|
| SEQ ID NO: 15 | GFTFSSYG |
| SEQ ID NO: 16 | IWYDGSNK |
| SEQ ID NO: 17 | ARRHYDFDAFDI |
| SEQ ID NO: 18 | SSDVGGYNY |
| SEQ ID NO: 19 | EVS |
| SEQ ID NO: 20 | SSYTSSSTLYV |
| SEQ ID NO: 21 | GGSISSSNW |
| SEQ ID NO: 22 | IYHSGNT |
| SEQ ID NO: 23 | ARDVSGGVNWFDP |
| SEQ ID NO: 24 | SGSIASNY |
| SEQ ID NO: 25 | EDN |
| SEQ ID NO: 26 | QSYDSSTVV |
| SEQ ID NO: 27 | GYSFTTFW |
| SEQ ID NO: 28 | IYPGDSDT |
| SEQ ID NO: 29 | ARSAVFDY |
| SEQ ID NO: 30 | QSVSSN |
| SEQ ID NO: 31 | GAS |
| SEQ ID NO: 32 | QQYNNWPTSWT |
| SEQ ID NO: 33 | GYSFTSYW |
| SEQ ID NO: 34 | IYPGDSD |
| SEQ ID NO: 35 | ARDYGDYYFDY |
| SEQ ID NO: 36 | SSDVGGYNY |
| SEQ ID NO: 37 | DVS |
| SEQ ID NO: 38 | CSYAGSYTGV |
| SEQ ID NO: 39 | GFTFDDYG |
| SEQ ID NO: 40 | INWSGADT |
| SEQ ID NO: 41 | AREVGIAVTGYWFDP |
| SEQ ID NO: 42 | HSES |
| SEQ ID NO: 43 | DDS |
| SEQ ID NO: 44 | QVWDSSSDHPGVV |
| SEQ ID NO: 45 | GYSFTSYW |
| SEQ ID NO: 46 | IYPGDSDT |
| SEQ ID NO: 47 | ARDYGDYYFDY |
| SEQ ID NO: 48 | SSDVGGYNY |
| SEQ ID NO: 49 | DVS |
| SEQ ID NO: 50 | CSYAGSYTGV |
| SEQ ID NO: 51 | GFTFDDYA |
| SEQ ID NO: 52 | ISWSSGSI |
| SEQ ID NO: 53 | AKDRASSWYAYGMDV |
| SEQ ID NO: 54 | SGSIASNY |
| SEQ ID NO: 55 | DDN |
| SEQ ID NO: 56 | QSTRV |

As an assay an ELISA (Enzyme-Linked Immunosorbent Assay) may be used for the quantitative measurement of human NMDAR1 autoantibodies in bodily fluids. The assay employs the antibody or antibody fragment or non-Ig scaffold specific for human NMDAR1 autoantibodies coated on a 96-well plate. Unspecific binding to the plate of proteins from the sample is prevented by pre-incubating the coated 96-well plate with a blocking solution (wash buffer with e.g. bovine serum albumin and a low concentration of detergent, e.g. Tween 20). 100 μl of standards and samples are pipetted into the wells and NMDAR1 antibodies present in a sample are bound to the wells via the immobilized antibody or antibody fragment or non-Ig scaffold during incubation (2.5 hours at room temperature or overnight at 4° C.). The wells are washed with washing buffer (e.g. with a solution of phosphate buffered saline) and diluted biotinylated anti-human IgG antibody is added and incubated for 1 hour at room temperature. After washing away unbound biotinylated antibody with washing buffer, diluted HRP-conjugated streptavidin is pipetted to the wells and incubated for 1 hour at room temperature. The wells are again washed with washing buffer, a substrate solution (e.g. tetra-methyl-benzidine) is added to the wells and colour develops in proportion to the amount of NMDAR1-IgG bound to the antibody or antibody fragment or non-Ig scaffold. Colour development is measured photometrically at a suitable wave length either directly or after adding a Stop Solution which stops the chemical colour reaction.

A sample of bodily fluid may be selected from the group comprising full blood, plasma, serum, cerebrospinal fluid (CSF), urine, saliva and amniotic fluid.

In a specific embodiment a sample of bodily fluid may be selected from the group comprising serum and CSF.

Subject matter of the present invention is a kit for determining the presence of anti-NMDAR1 antibodies in sample of a subject that may be in need of a therapy according to the present invention comprising:
1) A solid support with immobilized mixture of NMDAR1-binding antibodies or NMDAR1-binding antibody fragments or NMDAR1-binding non-Ig scaffolds
2) Washing buffer (bottles or powder for preparation) and dilution buffer.
3) Recombinant human NMDAR1 antibody for determination of standard curve.
4) Anti-human immunoglobulin antibody conjugated to a marker
5) In the case of enzymatic markers: Staining solution and Stop solution.

The solid support can be chosen depending on the device used for measurement. ELISA plates such as 96 well NUNC immunosorb plates are routinely used. Alternatives may be selected from particles such as beads or miniaturized plate formats such as microfluidic chips.

Washing buffers and blocking solutions for ELISA are known in the art. They consist of buffered saline solutions containing low detergent concentrations and/or saturation proteins that block unspecific sites of the ELISA plates. The buffers may be selected from the group comprising phosphate buffered saline or TRIS buffered saline. A commonly used detergent is Tween20 in the range of 0.5% to 10%. Saturation proteins may be selected from the group of skimmed milk, bovine serum albumin, serum or gelatin.

Dilution buffers may be identical to washing buffers or consist of saline buffered solutions only.

The anti-human immunoglobulin antibody may be selected from the group comprising anti-immunoglobulin G, anti-immunoglobulin A, anti-immunoglobulin M, anti-immunoglobulin D and anti-immunoglobulin E, e.g. polyclonal goat anti-Human IgG, IgM, IgA (H+L) Secondary Antibody (Life Technologies, Cat. #31128).

The marker can be either a reporter allowing quantification or a small molecule that interacts with a high affinity partner which is linked to a reporter. An example is the biotin-streptavidin system. Reporters known in the art are enzymes, e.g. horse radish peroxidase (HRP) or alkaline phosphatase, fluorophores or radioisotopes, A standard ELISA kit using the enzymes as a marker will also contain staining solution containing chromogenic substrates. For the enzyme HRP substrates may be selected from the group comprising TMB, DAB and ABTS. An acidic stop solution may be used to stop enzymatic activity before photometric measurement of optical density in standards and samples in order to determine concentrations of the protein or antibody of interest. Further embodiments within the scope of the present invention are set out below:
1) Antibody or antibody fragment or non-Ig scaffold binding specifically to a binding region of an anti-NMDAR1 antibody wherein the binding region of said anti-NMDAR1 antibody is comprised in one ore more sequences wherein said one or more sequence is selected from a group consisting of the following sequences:

(003-109-HC)
SEQ ID NO: 1
VQLVESGGGVVQPGRSLRLSCAASGFTFSSYGMHWVRQAPGKGLEWVAV
IWYDGSNKYYADSVKGRFTISRDNSKNTLYLQMNSLRAEDTAVYYCARR
HYDFDAFDIWGQGTMVTVSS
(003-109-LC)
SEQ ID NO: 2
QSALTQPASVSGSPGQSITISCTGTSSDVGGYNYVSWYQQHPGKAPKLM
IYEVSNRPSGVSNRFSGSKSGNTASLTISGLQAEDEADYYCSSYTSSST
LYVFGTGTKVTVL
(003-102-HC)
SEQ ID NO: 3
QVQLQESGPGLVKPSGTLSLTCAVSGGSISSSNWWSWVRQPPGKGLEWI
GEIYHSGNTNYNPSLKSRVTVSVDKSKNQFSLKLTSVTAADTAVYYCAR
DVSGGVNWFDPWGQGTLVTVSS
(003-102-LC)
SEQ ID NO: 4
NFMLTQPHSVSESPGKTVTISCTRSSGSIASNYVQWYQQRPGSAPTTVI
YEDNQRPSGVPDRFSGSIDSSSNSASLTISGLKTEDEADYYCQSYDSST
VVFGGGTKLTVL
(007-168-HC)
SEQ ID NO: 5
VQLVQSGAEAKKPGESLKISCKASGYSFTTFWIGWVRQMPGSGLEWIGI
IYPGDSDTRYSPSFQGHVTISADRSTSTAYLQWSSLKASDTAMYYCARS
AVFDYWGQGTLVTVSS
(007-168-LC)
SEQ ID NO: 6
EIVMTQSPATLSVSPGGRATLSCRASQSVSSNLAWYQQKPGQAPRLLIY
GASTRATGIPVRFSGSGSGTEFTLTISSLQSEDFAVYYCQQYNNWPTSW
TFGQGTKVEIK
(007-169-HC)
SEQ ID NO: 7
EVQLVQSGAEVKKPGESLKISCKGSGYSFTSYWIGWVRQMPGKGLEWMG
IIYPGDSDTRYSPSFQGQVTISADKSISTAYLQWSSLKASDTAMYYCAR
DYGDYYFDYWGQGTLVTVSS
(007-169-LC)
SEQ ID NO: 8
QSALTQPRSVSGSPGQSVTISCTGTSSDVGGYNYVSWYQQHPGKAPKLM
IYDVSKRPSGVPDRFSGSKSGNTASLTISGLQAEDEADYYCCSYAGSYT
GVFGEGTKLTVL
(007-124-HC)
SEQ ID NO: 9
EVQLVESGGGVGRPGGSLRLSCAASGFTFDDYGMSWVRQVPGKGLEWVS
GINWSGADTGYADSVKGRFTISRDNAKNSLYLQMNSLRVEDTALYHCAR
EVGIAVTGYWFDPWGQGTLVTV
(007-124-LC)
SEQ ID NO: 10
SYELTQPPSVSVAPGQTARISCGGNHSESVHWYQQKPGQAPVLVVYDDS
DRPSGIPERFSGSKSGNTATLTISRVEGGDEAEYYCQVWDSSSDHPGVV
FGGGTKLTVL
(007-142-HC)
SEQ ID NO: 11
EVQLVQSGAEVKKPGESLKISCKGSGYSFTSYWIGWVRQMPGKGLEWMG
IIYPGDSDTRYSPSFQGQVTISADKSISTAYLQWSSLKASDTAMYYCAR
DYGDYYFDYWGQGTLVTVSS
(007-142-LC)
SEQ ID NO: 12
LTQPRSVSGSPGQSVTISCTGTSSDVGGYNYVSWYQQHPGKAPKLMIYD
VSKRPSGVPDRFSGSKSGNTASLTISGLQAEDEADYYCCSYAGSYTGVF
GGGTKLTVL
(008-218-HC)
SEQ ID NO: 13
EVQLVESGGGLVQPGRSLRLSCAASGFTFDDYAMHWVRQVPGKGLEWVS
GISWSSGSIGYADSVKGRFTISRDNAKNSLYLQMNSLRAEDTALYYCAK
DRASSWYAYGMDVWGQGTLVTV
(008-218-LC)
SEQ ID NO: 14
NFMLTQPHSVSESPGKTVTISCTRSSGSIASNYVQWYQQRPGSSPTTVI
YDDNQRPSGVPNRFSGSIDSSSNSASLIISGLKTEDEADYYCQSTRVFG
GGTKLTVL

and wherein said binding region for the antibody or antibody fragment or non-Ig scaffold comprises or consists of one or several of the below mentioned sequences:

| | |
|---|---|
| SEQ ID NO: 15 | GFTFSSYG |
| SEQ ID NO: 16 | IWYDGSNK |
| SEQ ID NO: 17 | ARRHYDFDAFDI |
| SEQ ID NO: 18 | SSDVGGYNY |
| SEQ ID NO: 19 | EVS |
| SEQ ID NO: 20 | SSYTSSSTLYV |
| SEQ ID NO: 21 | GGSISSSNW |
| SEQ ID NO: 22 | IYHSGNT |
| SEQ ID NO: 23 | ARDVSGGVNWFDP |
| SEQ ID NO: 24 | SGSIASNY |
| SEQ ID NO: 25 | EDN |
| SEQ ID NO: 26 | QSYDSSTVV |
| SEQ ID NO: 27 | GYSFTTFW |
| SEQ ID NO: 28 | IYPGDSDT |
| SEQ ID NO: 29 | ARSAVFDY |
| SEQ ID NO: 30 | QSVSSN |
| SEQ ID NO: 31 | GAS |
| SEQ ID NO: 32 | QQYNNWPTSWT |
| SEQ ID NO: 33 | GYSFTSYW |
| SEQ ID NO: 34 | IYPGDSD |
| SEQ ID NO: 35 | ARDYGDYYFDY |
| SEQ ID NO: 36 | SSDVGGYNY |
| SEQ ID NO: 37 | DVS |
| SEQ ID NO: 38 | CSYAGSYTGV |
| SEQ ID NO: 39 | GFTFDDYG |
| SEQ ID NO: 40 | INWSGADT |
| SEQ ID NO: 41 | AREVGIAVTGYWFDP |
| SEQ ID NO: 42 | HSES |
| SEQ ID NO: 43 | DDS |
| SEQ ID NO: 44 | QVWDSSSDHPGVV |
| SEQ ID NO: 45 | GYSFTSYW |
| SEQ ID NO: 46 | IYPGDSDT |
| SEQ ID NO: 47 | ARDYGDYYFDY |
| SEQ ID NO: 48 | SSDVGGYNY |
| SEQ ID NO: 49 | DVS |
| SEQ ID NO: 50 | CSYAGSYTGV |
| SEQ ID NO: 51 | GFTFDDYA |
| SEQ ID NO: 52 | ISWSSGSI |
| SEQ ID NO: 53 | AKDRASSWYAYGMDV |
| SEQ ID NO: 54 | SGSIASNY |
| SEQ ID NO: 55 | DDN |
| SEQ ID NO: 56 | QSTRV |

2) Antibody or antibody fragment or non-Ig scaffold binding to a binding region of an anti-NMDAR1 antibody according to claim 1 wherein said antibody or antibody fragment or non-Ig scaffold binding to a binding region of an anti-NMDAR1 antibody is a non-IgG scaffold.
3) Antibody or antibody fragment or non-Ig scaffold binding to a binding region of an anti-NMDAR1 antibody according to claim 1 or 2 wherein said non-Ig scaffold may be selected from the group comprising tetranectin-based non-Ig scaffold, fibronectin scaffold, lipocalin-based scaffold, ubiquitin scaffolds, transferrin scaffolds, protein A scaffolds, ankyrin repeat based scaffolds, microproteins, preferably microproteins forming a cysteine knot, scaffolds, Fyn SH3 domain based scaffolds, EGFR-A-domain based scaffolds and Kunitz domain based scaffolds and aptamers.
4) Non-Ig scaffold binding to a binding region of an anti-NMDAR1 antibody according to claim 3 wherein said non-Ig scaffold is an oligonucleotide aptamer.
5) Antibody or antibody fragment or non-Ig scaffold binding to a binding region of an anti-NMDAR1 antibody according to any of claims 1 to 4 wherein said antibody or antibody fragment or non-Ig scaffold exhibits an affinity towards said binding region of an anti-NMDAR1 antibody in such that the dissociation constant K_{D} is lower than 10⁻⁷ M, preferred 10⁻⁸ M, preferred K_{D} is lower than 10⁻⁹ M, most preferred lower than 10⁻¹⁰ M with said binding region of the anti-NMDAR1 antibody.
6) Antibody or antibody fragment or non-Ig scaffold binding to a binding region of an anti-NMDAR1 antibody according to any of claims 1 to 5 wherein said antibody or antibody fragment or non-Ig scaffold neutralizes the anti-NMDAR1 antibodies.
7) Antibody or antibody fragment or non-Ig scaffold binding to a binding region of an anti-NMDAR1 antibody according to any of claims 1 to 6 wherein said antibody or antibody fragment or non-Ig scaffold is a monospecific antibody or antibody fragment or non-Ig scaffold.
8) Antibody or antibody fragment or non-Ig scaffold binding to a binding region of an anti-NMDAR1 antibody according to any of claims 1 to 7 for use in therapy of a disease or a condition in a subject in need of such a therapy said disease or condition being associated with anti-NMDAR1 antibodies.
9) Antibody or antibody fragment or non-Ig scaffold binding to a binding region of an anti-NMDAR1 antibody according to any of claims 1 to 7 for use in therapy of a disease or a condition of said disease or condition being associated with or caused by anti-NMDAR1 antibodies in a subject in need of such a therapy, if said subject exhibits the presence of anti-NMDAR1 antibodies in a bodily fluid and exhibits at least one clinical symptom or clinical condition selected from the group comprising the clinical symptoms/conditions according to the following list (ICD numbers in parentheses refer to the WHO International Classification of Diseases which defines the clinical conditions):
   psychiatric abnormalities including depression (F32), mania with psychotic symptoms (F30.2), anxiety (F06.4), phobic anxiety (F40), delusions (F22.0), obsessive-compulsive disorder (F42), organic delusional disorder (F06.3), catatonia (F06.1, F20.2), acute polymorphic psychotic disorder (F23.0, F23.1), dissociative disorders (F44)
   movement disorders including dyskinesias/dystonia (G24), myoclonus (G25.3), tremor (G25.0, G25-1, G25-2), tics (F95, G25.69)
   epileptic seizures (G40)
   hypoventilation (R06.89)
   mild cognitive impairment (F06.7)
   dementia in Alzheimer's disease (F00), vascular dementia (F01), dementia in other diseases (F02)
   pregnancy.
10) Antibody or antibody fragment or non-Ig scaffold binding to a binding region of an anti-NMDAR1 antibody according to any of claims 1 to 7 for use in therapy of a disease or a condition in a subject said disease or condition being associated with anti-NMDAR1 antibodies according to claim 8 or 9 wherein said antibody or antibody fragment or non-Ig scaffold is administered in vivo to said subject in need thereof.
11) Antibody or antibody fragment or non-Ig scaffold binding to a binding region of an anti-NMDAR1 antibody according to any of claims 1 to 7 for use in therapy of a disease or a condition in a subject said disease or condition being associated with anti-NMDAR1 antibodies according to any of claims 8 to 10 wherein said antibody or antibody fragment or non-Ig scaffold is administered to said subject in need thereof intravenously.
12) Antibody or antibody fragment or non-Ig scaffold binding to a binding region of an anti-NMDAR1 antibody according to any of claims 1 to 7 for use in therapy of a disease or a condition in a subject said disease or condition being associated with anti-NMDAR1 antibodies according to any of claims 8 to 11 wherein said antibody or antibody fragment or non-Ig scaffold is used in an ex vivo therapy of said subject in need thereof.
13) Antibody or antibody fragment or non-Ig scaffold binding to a binding region of an anti-NMDAR1 antibody according to any of claims 1 to 7 for use in therapy of a disease or a condition in a subject said disease or condition being associated with anti-NMDAR1 antibodies according to any of claims 8 to 12 wherein said subject exhibits the presence of anti-NMDAR1 antibodies when measured according to a method of Example 1.2.
14) Antibody or antibody fragment or non-Ig scaffold binding to a binding region of an anti-NMDAR1 antibody according to any of claims 1 to 7 for use in therapy of a disease or a condition in a subject said disease or condition being associated with anti-NMDAR1 antibodies according to any of claims 8 to 13 wherein said antibody or antibody fragment or non-Ig scaffold is to be used in combination with a chemotherapeutic agent or a immunosuppressive agent.
15) A method of treatment or prevention of a disease or medical condition associated with anti-NMDAR1 antibodies according to any of claims 8 to 14, which comprises the administration of an effective amount of antibody or antibody fragment or non-Ig scaffold binding to a binding region of an anti-NMDAR1 antibody according to any of claims 1 to 7 to a subject in need thereof.
16) Use of a compound according to any of claims 1 to 7, in the manufacture of a medicament for the treatment or prevention of a disease or medical condition associated with anti-NMDAR1 antibodies according to any of claims 8 to 14.
17) Antibody or antibody fragment or non-Ig scaffold-coated device for plasma exchange or CSF exchange (liquorpheresis) wherein said antibody or antibody fragment or non-Ig scaffold is an antibody or antibody fragment or non-Ig scaffold according to any of claims 1 to 7.
18) Antibody or antibody fragment or non-Ig scaffold-coated device for plasma exchange or CSF exchange (liquorpheresis) according to claim 17 wherein said antibody or antibody fragment or non-Ig scaffold-coated device is an antibody or antibody fragment or non-Ig scaffold-coated column.
19) Use of an antibody or antibody fragment or non-Ig scaffold according to any of claims 1 to 7 for determining the presence of anti-NMDAR1 antibodies in a sample of a bodily fluid of a subject having a disease or condition being associated with anti-NMDAR1 antibodies.
20) A method of determining the presence of an anti-NMDAR1 antibody in a sample of bodily fluid of a subject in order to determine whether said subject is in need of a therapy wherein said subject has a disease or condition being associated with anti-NMDAR1 antibodies and the method comprising:
   contacting a sample of bodily fluid obtained from said subject with at least one antibody or antibody fragment or non-Ig scaffold according to any of claims 1 to 7
   determining the presence of said anti-NMDAR1 antibodies in said sample of bodily fluid
   wherein in case an anti-NMDAR1 antibody is present in said sample said subject may have a disease or condition associated with anti-NMDAR1 antibodies and may be in need of a therapy.
21) Kit for determining the presence of anti-NMDAR1 antibodies in sample of a subject that may be in need of a therapy comprising:
   1) A solid support with immobilized mixture of NMDAR1-binding antibodies or NMDAR1-binding antibody fragments or NMDAR1-binding non-Ig scaffolds
   2) Recombinant human NMDAR antibody for determination of standard curve.
   3) Anti-human immunoglobulin antibody conjugated to a quantifiable marker

### EXAMPLES

### Example 1

1.1 Generation of Monoclonal Human Recombinant NMDAR1 Antibodies

(Technical procedure based on Tiller et al. 2009, J Immunol Methods 350(1-2):183-93)

Isolation of Single Human Plasma Cells and Memory B Cells from Cerebrospinal Fluid samples (FIG. 3-4)

Cerebrospinal fluid samples (CSF) were collected in the context of the general routine examination after signed informed consent in accordance with Charité ethics board approval. CSF samples were centrifuged at 400×g for 10 minutes. Then supernatant was decanted and cells were suspended in 500 μl of freezing medium (45% RPMI, 45% FCS, 10% DMSO) to be stored at −80° C. until further use. For the fluorescence activated cell sorting (FACS), frozen cells were thawed, diluted and stained on ice with the antibodies as shown in FIGS. 3-4. Cell sorting was performed on an FACSAria II (BD Biosciences) into 96-well PCR plates (VWR) containing 4 μl/well of ice-cold lysis solution of 0.5× phosphate-buffered saline (PBS) with 10 mM DTT (Invitrogen), 8 U RNAsin (Promega). After the sort, plates were directly sealed with Sealing Foil (VWR) and immediately frozen on dry ice before storage at −80° C.

Single Cell Reverse Transcription PCR and Amplification of Ig Genes

The reverse transcription (RT) was performed in the original 96-well sorting plate in a total volume of 14 μl per sample. To the total RNA from each single cell, to each well 150 ng random hexamer primer p(dN)₆ (Roche), 0.5 μl of 25 mM from each nucleotide dNTP-Mix (Invitrogen), 1 μl 0.1 M DTT (Invitrogen), 0.5 μl 10% Igepal CA-630 (Sigma), 14 U RNAsin (Promega) and 50 U Superscript® III reverse transcriptase (Invitrogen) were added. Thermal cycling conditions were 42° C. for 10 min, 25° C. for 10 min, 50° C. for 60 min and 94° C. for 5 min. cDNA was stored at −20° C.

For Ig V gene amplification a nested PCR strategy in two steps was used, for each single cell cDNA separately for IgH, Igκ and Igλ. All PCR reactions were performed in 96-well plates (VWR) in a total volume of 40 μl per well containing 320 nM of total primer or primer mix, 250 nM each dNTP (Invitrogen) and 0.9 U HotStar® Taq DNA polymerase (Qiagen). As templates for first PCR's 2.0 μl of cDNA were used, for nested reactions 3.5 μl of unpurified first PCR product. Each round of PCR was performed at initial 94° C. for 15 min, 50 cycles at 94° C. for 30 sec, 58° C. (IgH/Igκ) or 60° C. (Igλ) for 30 sec and 72° C. for 55 sec (1st PCR) or 45 sec (2nd PCR) before final 72° C. for 10 min.

Ig Gene Sequence Analysis

The second PCR products were sequenced with the respective reverse primer for IgH, Igκ bzw. Igλ as outlined in Tiller et al. 2009. J Immunol Methods 350(1-2):183-93. Sequences were analyzed by IgBLAST comparison with GenBank (Ye J et al. 2013. Nucleic Acids Res. 41)) to identify germline V(D)J gene segments with highest identity. IgH complementarity determining region (CDR)3 length was determined as indicated in IgBLAST by counting the amino acid residues following framework region (FWR)3 up to the conserved tryptophan-glycine motif in all JH segments or up to the conserved phenylalanin-glycine motif in JL segments. In contrast to sequences from cloned Ig genes, 2nd PCR product sequences are unlikely to show the mutations that were introduced by the Taq polymerase and would do so only if the mutations were introduced early during the PCR. Analysis of Ig gene sequences from naive B cells lacking somatic mutations allows the detection of Taq-mediated misincorporated nucleotides by comparison to published germline sequences.

Expression Vector Cloning

Before cloning, all PCR products were purified using Qia-Quick 96 PCR Purification Kit (Qiagen) and QIAvac96. Samples were eluted with 50 μl nuclease-free water (Eppendorf) into 96-well plates. Digests were carried out with the respective restriction enzymes AgeI, SalI and XhoI (all from NEB) in the same plate in a total volume of 35-40 μl and digested PCR products were purified before ligation into human Igγ1, Igκ and Igλ, expression vectors containing an Ig gene signal peptide sequence (GenBank accession no. DQ407610) and a multiple cloning site upstream of the human Igγ1, Igκ or Igλ, constant regions. Transcription is under the influence of the human cytomegalovirus (HCMV) promotor and clones can be selected based on resistance to ampicillin. Ligation was performed in a total volume of 10 μl with 1 U T4-Ligase (Invitrogen), 7.5 μl of digested and purified PCR product and 25 ng linearized vector. Competent E. coli DH10B bacteria (Clontech) were transformed at 42° C. with 3 μl of the ligation product in 96-well plates. Colonies were screened by PCR using 5′ Absense as forward primer and 3′ IgGinternal, 3′ Cκ494 or 3′ Cλ, as reverse primer, respectively. PCR products of the expected size (650 bp for Igγ1, 700 bp for Igκ and 590 bp for Igλ) were sequenced to confirm identity with the original PCR products. (Tiller et al. 2009, J Immunol Methods 350(1-2):183-93). Due to the use of error-prone Taq-Polymerase approximately Plasmid DNA was isolated from 3 ml bacteria cultures grown for 16 h at 37° C. in Terrific Broth (Difco Laboratories) containing 75 μg/ml ampicillin (Sigma) using QIAprep Spin columns (Qiagen). From 1.5 ml baceria cultures, on average 35 μg plasmid DNA was recovered after elution with 75 μl of EB elution buffer (Qiagen).

Recombinant Antibody Production

Human embryonic kidney (HEK) 293 (ATCC, No.CRL-1573) or 293T (ATCC, No. CRL-11268) cells were cultured in 150 mm plates (Falcon, Becton Dickinson) under standard conditions in Dulbecco's Modified Eagle's Medium (DMEM; GibcoBRL) supplemented with 10% heat-inactivated ultralow IgG fetal calf serum (FCS) (Invitrogen), 1 mM sodium pyruvate (GibcoBRL), 100 μg/ml streptomycin, 100 U/ml penicillin G and 0.25 μg amphotericin (all from GibcoBRL).

Transient transfections of exponentially growing HEK293 cells were performed by calciumphosphate precipitation at 80% cell confluency. Equal amounts (12.5-20 μg each) of IgH and corresponding IgL chain expression vector DNA and 0.7 mM chloroquine (Sigma) were mixed in 1 ml sterile water and 2.5 M CaCl2 was added drop-wise to a concentration of 250 mM. An equal volume of 2×HEPES-buffered saline (50 mM HEPES, 10 mM KCl, 12 mM Dextrose, 280 mM NaCl, 1.5 mM Na2HPO4-7H2O, pH 7.05) was mixed with the calcium-DNA solution under slow vortexing and incubated at room temperature for 10 min to allow formation of precipitates. The precipitation mixture was distributed evenly to the culture dish. The cells were washed with 10 ml serum-free DMEM after 8-12 h and cultured for 6 d in 25 ml DMEM supplemented with 1% Nutridoma-SP (Roche) before supernatants were harvested and analyzed by enzyme-linked immunosorbent assay (ELISA) for recombinant antibody production.

Recombinant Antibody Purification

Cell debris was removed by centrifugation at 800×g for 10 min and culture supernatants were stored at 4° C. with 0.05% sodium azide. Recombinant antibodies were purified with Protein G beads (GE Healthcare) according to the manufacturer's instructions. In brief, 25 ml cell culture supernatant was incubated with 25 μl Protein G beads for at least 14 h at 4° C. under rotation. Supernatants were removed after centrifugation at 800×g for 10 min and the beads were transferred to a chromatography spin column (BioRad) equilibrated with PBS. After two rounds of washing with 1 ml PBS, antibodies were eluted in 3-4 fractions (200 μl each) with 0.1 M glycine (pH 3.0). Eluates were collected in tubes containing 20 μl 1 M Tris (pH 8.0) with 0.5% sodium azide. Recombinant antibody concentrations were determined by ELISA, all steps were performed at ambient temperature.

1.2. Validation of Antibody Binding to Human NMDAR1 Protein and Pathogenic Effects

Transfected HEK293 Cells (FIG. 5)

The cDNA of the human ionotropic glutamate N-methyl-D-aspartate 1 receptor (GRIN1) was kindly provided by Prof. Dr. Wanker (MDC, Berlin) and cloned into pBudCE4.1 (Life Technologies). NR1 DNA (1 μg) was mixed with 3 μg PEI and 100 μl 150 mM NaCl, vortexed and incubated for 10 min, and HEK293 cells were transiently transfected. Two days later, HEK293 cells on cover slips were fixed with methanol at −20° C. for 4 min. In addition, HEK293 cells transfected with a different NMDAR clone, leucine-rich glioma-inactivated 1 (LGI1), contactinassociated protein 2 (Caspr2), α-amino-3-hydroxy-5-methyl-4-isoxazolepropionic acid (AMPA) receptor, and gamma-aminobutyric acid b (GABAb) receptor were used (Autoimmune-Enzephalitis-Mosaik 1, Euroimmun, Lübeck, Germany). For staining with monoclonal antibodies and control antibodies, cells were washed in PBS, preincubated with 5% normal goat serum containing 2% bovine serum albumin and 0.1% Triton X-100, and incubated with antibodies starting at a 1:2 dilution of the cell culture supernatant overnight at 4° C. Secondary fluorescently labeled anti-human IgG antibodies were used for visualization. Sections were washed in PBS and coverslips mounted with Immu-Mount (ThermoScientific). Double-labeling of transfected cells was performed using commercial antibodies: monoclonal mouse and polyclonal rabbit anti-NR1 (1:100, Synaptic Systems) (FIG. 5) and reactive clones were identified.

The following reactive clones were identified (HC=heavy chain; LC=light chain; bold=antigen binding regions (CDR1-3) of the respective chains): Seq.-ID 1-14.

Brain Sections (FIG. 6)

Paraformaldehyde-fixed mouse and rat brain sections were used. Tissue was permeabilized in 0.1% Triton X-100 in PBS for 20 min and blocked in 10% normal goat serum for 30 min. Culture supernatants of transfected HEK293 cells containing monoclonal human recombinant or control antibodies were diluted 1:2 to 1:200 and sections incubated overnight at 4° C. Secondary fluorescently labeled anti-human IgG antibodies were used for visualization. Sections were washed in PBS and coverslips mounted with Immu-Mount (ThermoScientific).

Staining pattern of NR1-reactive clones was identical to the known anatomical distribution of NMDAR in the mouse hippocampus (FIG. 6).

Down-Regulation of NMDAR-Positive Synaptic Clusters

Primary hippocampal neurons were cultured after dissection from mouse brains. Hippocampi at embryonic day 16 were dissociated in MEM supplemented with 10% fetal calf serum, 100IE insulin/l, 0.5 mM glutamine, 100 U/ml penicillin/streptomycin, 44 mM glucose and 10 mM HEPES. Following centrifugation, cells were resuspended in serum-free neurobasal medium supplemented with B27, 0.5 mM glutamine, 100 U/ml penicillin/streptomycin and 25 μM glutamate and 8×10⁴ cells/well plated on cover slips precoated with poly-L-lysine/collagen (all ingredients from Gibco/BRL). Cells were used for immunocytochemistry at day 14 in vitro to allow for full maturation of functional synapses.

For quantification of NMDAR-positive synaptic clusters, primary neurons were treated for 18 hours with monoclonal human recombinant anti-NMDAR1 antibody or control antibody (FIG. 7). After incubation with pathogenic and control antibodies, the cells were fixed and stained for the non-internalized fraction of NMDAR1s using commercial NMDAR antibodies (Synaptic Systems). Clusters were determined using 40 images of 100 μm proximal dendrite length at 40× magnification per condition in each individual experiment. Images were converted to greyscale. Color-inverted and thresholded images were analyzed by Scion Image software (Scion, now http://en.bio-soft.net/) according to intensity and size criteria. For accurate comparison between treatment groups regarding areas of similar cell numbers, cell counts were performed. Down-regulation of NMDAR clusters with the monoclonal human NMDAR antibody (FIG. 7) is comparable to the data in the literature using whole CSF of NMDAR encephalitis patients.

Epitope Analysis

Point mutation N368Q was introduced into the NR1 construct using the Stratagene QuikChange Mutagenesis kit according to manufacturer's instructions and the mutant transiently transfected into HEK293 cells as described previously (Doss et al, 2014). Staining of HEK293 cells expressing natural and mutated NR1 construct was performed as described above. Binding to the mutant was eliminated for all monoclonal human NMDAR1 antibodies (FIG. 8).

### Example 2

Generation of Antibodies Against Monoclonal Human Recombinant NMDAR1 Antibodies

A scFv library was constructed from patient PBMC as described in Frenzel et al. 2014. Methods Mol Biol. 1060:215-43. Panning was performed over three rounds on immobilized human monoclonal anti-NMDAR1-antibody and screening was performed by ELISA on immobilized antigen and myc-Tag-detection as described in Hust et al. 2014 Methods Mol Biol. 1101:305-20.

### Example 3

Generation of Aptamers Against Monoclonal Human Recombinant NMDAR1 Antibodies

The generation of aptamers is conducted according to Jones et al. 2006. Antimicrob. Agents Chemother. 50(9): 3019-3027. SELEX (Ellington and Szostak 1990. Nature. 346(6287):818-22; Tuerk and Gold 1990. Science. 249(4968):505-10) was used to select for aptamers that recognize human monoclonal anti-NMDAR1-antibodies attached to cyanogen bromide (CNBr)-activated Sepharose via an N-terminal linker OR via Protein-A-Sepharose. A DNA library with a diversity of 10¹⁴ comprising a 40-nt random region flanked by two primer binding sites was in vitro transcribed to yield the respective RNA library. RNA was incubated with the selection matrix and after removal of nonbinding sequences by washing with binding buffer, remaining species were eluted, reverse-transcribed, and used as input DNA for the next transcription and a new selection cycle. Binding species were enriched after six cycles of selection, reverse-transcribed, cloned, and sequenced. Monoclones exhibiting better binding properties in column assays to anti-NMDAR1-antibody-Sepharose than the enriched pool from cycle 6 were selected for affinity determination.

### Example 4

Assay for Determining the Binding Affinity of the Antibodies and Aptamers Against Monoclonal Human Recombinant NMDAR1 Antibodies

The affinity of selected antibodies or aptamers was measured by surface plasmon resonance (SPR) analysis (Jones et al. 2006. Antimicrob. Agents Chemother. 50(9):3019-3027). In more detail, the Biacore™ X platform (GE Healthcare) was used to perform binding analysis of the selected aptamers. Therefore, monoclonal NMDAR antibodies are immobilized onto protein A sensor chip (GE Healthcare) utilizing amine coupling as described (Schütze T, et al. 2011, PLoS ONE 6(12): e29604.). Binding analysis is conducted at a flow rate of 30 μl/min with binding buffer at 25° C. Prior to injection, synthetic oligonucleotides are denatured for 3 min at 94° C. and refolded in binding buffer. 30 μl of the aptamer solution in a range from 0.1 to 2.0 μM are injected into the flow cell. After each aptamer injection, the chip surface was regenerated by injection of 2×10 μl 0.5 mM NaCl/0.5 mM MgCl₂. Association and dissociation rates and constants of the aptamer-streptavidin complexes are determined using BIAevaluation software (Biacore).

### Example 5

5.1. Inhibition of Binding (“Neutralization”) of Monoclonal Human Recombinant NMDAR1 Antibodies to NMDAR1-Expressing HEK293 Cells
HEK293 cells were transiently transfected with the cDNA of the human ionotropic glutamate N-methyl-D-aspartate 1 receptor (Gene ID: GRIN1) and grown on coverslips for immunocytochemistry as described above. Specific aptamers or Ig or non-Ig scaffold were pre-incubated with monoclonal anti-NMDAR1 antibodies for 30 min at room temperature at 2-20 fold molar excess of aptamer vs anti-NMDR1 antibody. Controls contained either monoclonal antibody, non-anti-NMDAR1 control antibodies or aptamer only. Cells were washed in PBS, preincubated with 5% normal goat serum containing 2% bovine serum albumin and 0.1% Triton X-100 and incubated with aptamer-antibody mixtures or control antibodies overnight at 4° C. Secondary fluorescently labeled anti-human IgG antibodies were used for visualization. Coverslips were washed in PBS and mounted with Immu-Mount (ThermoScientific). Neutralization of antibody binding is determined by reduction of fluorescence signal to baseline levels, i.e. fluorescence intensity of cells incubated with the non-NMDAR binding control antibody.

5.2. Inhibition of Binding of Monoclonal Human Recombinant NMDAR1 Antibodies to NMDAR1-Expressing Brain Sections
Paraformaldehyde-fixed mouse and rat brain sections were used. Tissue was permeabilized in 0.1% Triton X-100 in PBS for 20 min and blocked in 10% normal goat serum for 30 min. Specific aptamers or blocking Ig or non-Ig scaffold were pre-incubated with anti-NMDAR1 monoclonal antibodies for 30 min at room temperature at 2-20 fold molar excess of aptamer vs anti-NMDR1 antibody. Controls contained either monoclonal antibody, non-NMDAR control antibodies or aptamer only. Sections were incubated overnight at 4° C. Secondary fluorescently labeled anti-human IgG antibodies were used for visualization. Sections were washed in PBS and coverslips mounted with Immu-Mount (ThermoScientific). The staining pattern was compared to the known anatomical distribution of NMDAR in the mouse hippocampus. Neutralization of antibody binding is determined by reduction of fluorescence signal to baseline levels, i.e. fluorescence intensity of sections incubated with the non-NMDAR binding control antibody (FIG. 11). Pre-incubation of anti-NMDAR monoclonal antibody with aptamer markedly reduces the binding of the anti-NMDAR monoclonal antibody to the NMDAR within the dentate gyrus of the hippocampus.

5.3. Inhibition of Auto-Antibody-Mediated Downregulation of NMDAR-Positive Postsynaptic Clusters
Primary hippocampal neurons were cultured and prepared for immunocytochemistry as described above. Cells were used at day 14 in vitro to allow full maturation of functional synapses. NMDAR-positive synaptic clusters on primary neurons were quantified as before comparing the staining by monoclonal human recombinant anti-NMDAR1 antibody with the pre-incubated aptamer-antibody mixture (molar ratio 20:1).

5.4. Inhibition of Binding of NMDAR1-Positive Patient Serum Antibodies to NMDAR1-Expressing HEK293 Cells
HEK293 cells were transiently transfected with the cDNA of the human ionotropic glutamate N-methyl-D-aspartate 1 receptor (GeneID: GRIN1) and grown on coverslips for immunocytochemistry as described above. Specific aptamers were pre-incubated with patient serum for 30 min at room temperature. Controls contained either control antibodies or patient serum only. Cells were washed in PBS, preincubated with 5% normal goat serum containing 2% bovine serum albumin and 0.1% Triton X-100 and incubated with sample mixtures or controls overnight at 4° C. Secondary fluorescently labeled anti-human IgG or IgA antibodies were used for visualization. Coverslips were washed in PBS and coverslips mounted with Immu-Mount (ThermoScientific). The fluorescence intensity of cells was recorded. Inhibition was determined by significant signal reduction in samples containing aptamers compared to samples with patient serum only.

### Example 6: Sequence Analysis of Patient-Derived NMDAR-Autoantibodies

Patient-derived NMDAR-autoantibodies were isolated as described in Example 1. Sequences were analyzed by IgBLAST comparison with GenBank (Ye J et al. 2013. Nucleic Acids Res. 41). The CDRs were identified, aligned and analysed for CDR lengths, properties of CDR residues and sequence homology (FIG. 9). CDR sequence alignment revealed functional homology human NMDAR antibodies derived from different patients.

### Example 7: Identification of Unmutated Human NMDAR-Autoantibodies (Kreye et al. 2016, Brain)

For each IgG sequence the number of somatic hypermutations in the immunoglobulin gene was counted in comparison to the annotated germline sequences as well as the length of the complementarity determining regions (Kabat and Wu, 1991; Kabat et al. 1983). Unmutated human antibodies against the NMDAR were identified (FIG. 10). These antibodies comprise the so-called germ-line configuration (also called ‘naturally occurring antibodies’), i.e. they are continuously generated by the body, not only in patients but in everyone and will thus stochastically be present also in previously healthy persons. Due to the sequence code of naturally occurring NMDAR antibodies in the normal (healthy) genetic repertoire, the data suggest that there is an evolutionary restriction to a limited number of sequences which might be relevant to larger numbers of people.

### FIGURE DESCRIPTION

FIG. 1a:

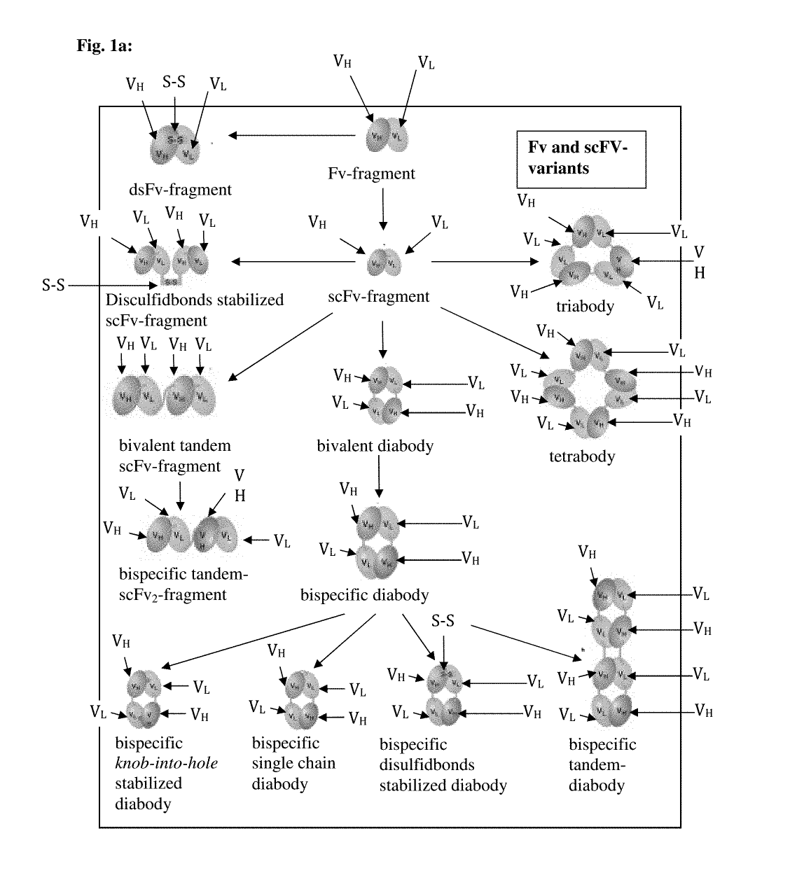

Illustration of antibody formats—Fv and scFv-Variants

FIG. 1b:

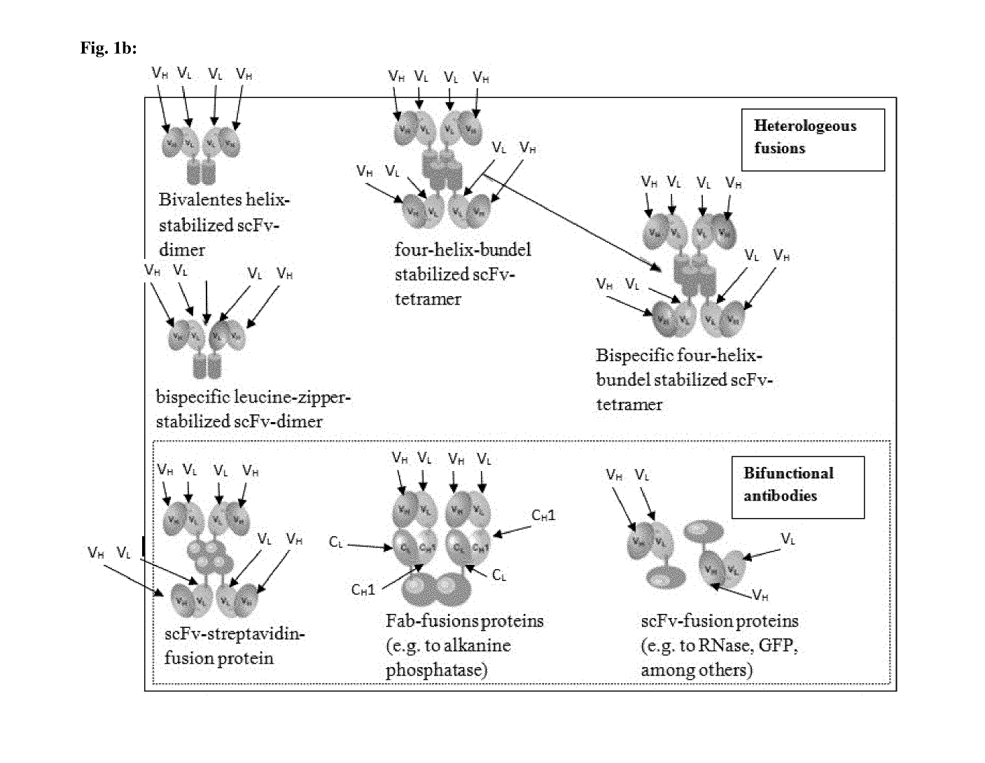

Illustration of antibody formats—heterologous fusions and bifunctional antibodies

FIG. 1c:

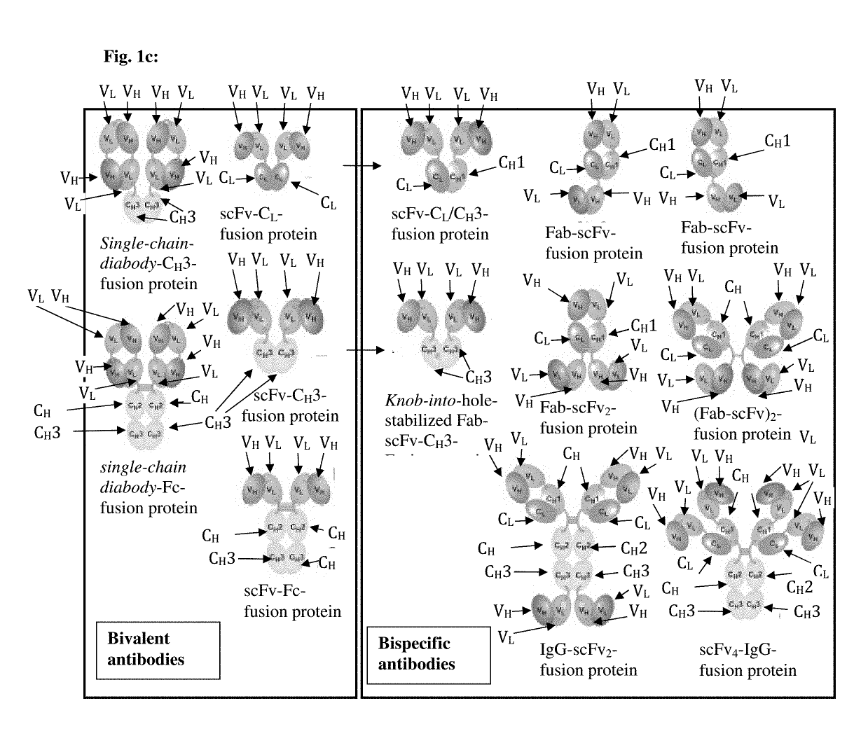

Illustration of antibody formats—bivalent antibodies and bispecific antibodies

FIG. 2:

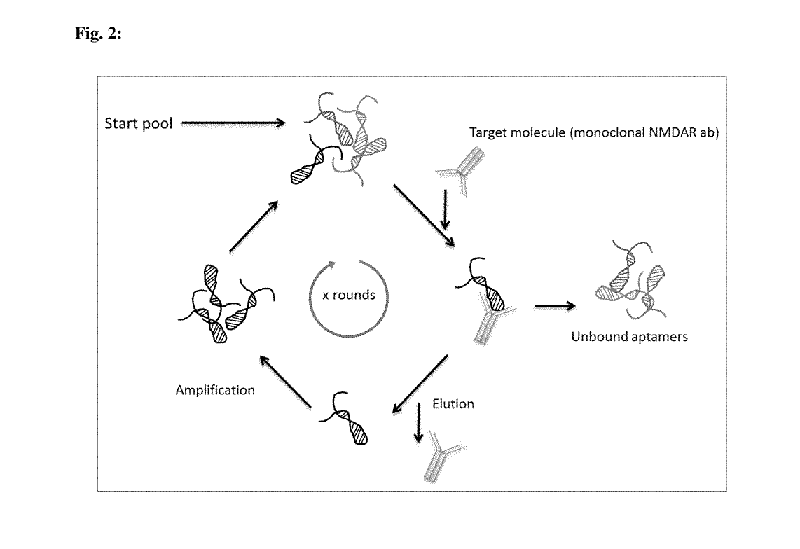

Several rounds of selection and amplification result in highly specific and affine aptamers

FIG. 3:

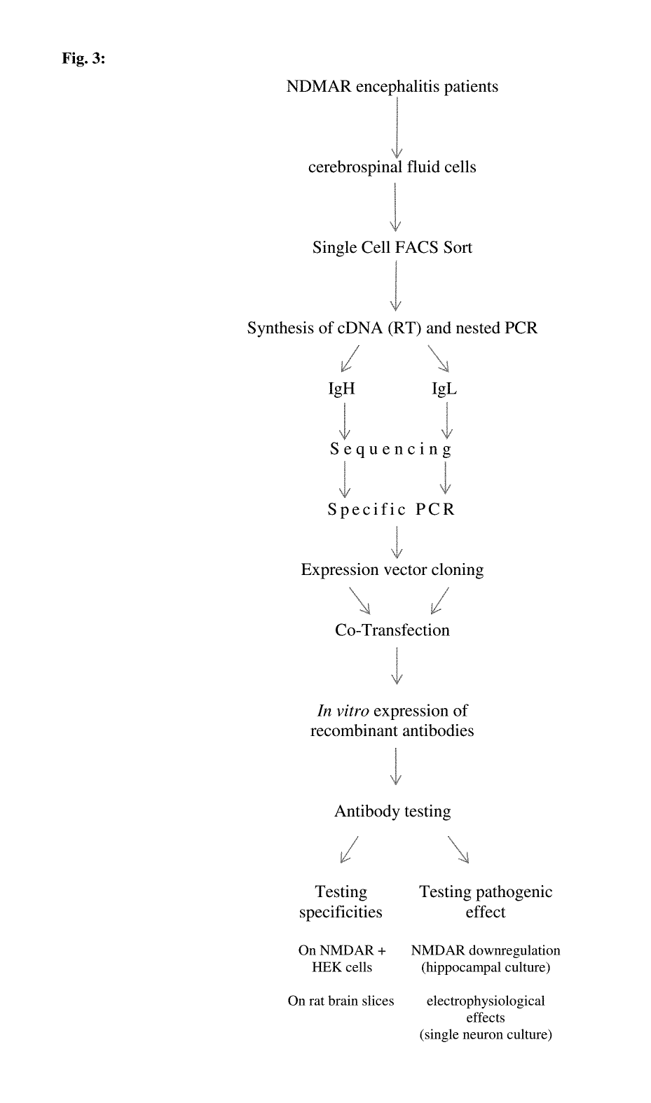

First monoclonal recombinant NMDAR1 autoantibody

Technical overview

FIG. 4:

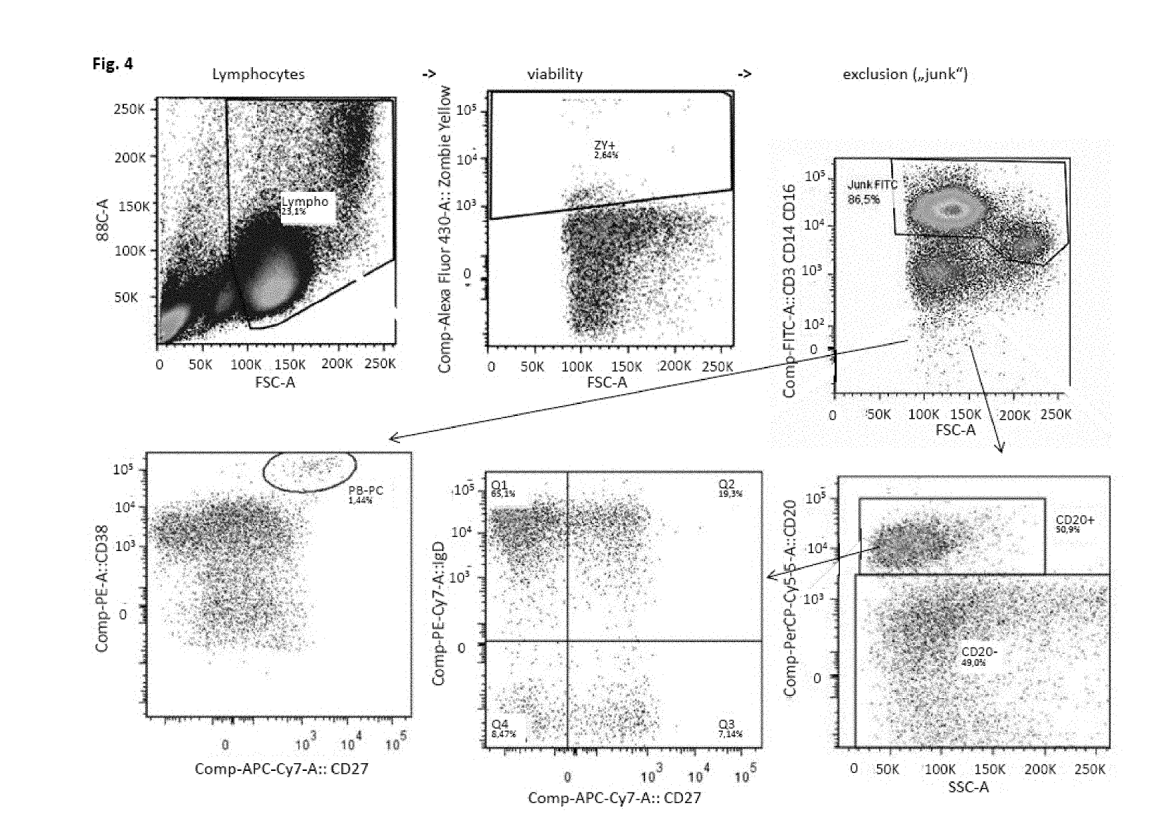

First monoclonal recombinant NMDAR1 autoantibody

FACS Sort Strategy

FIG. 5:

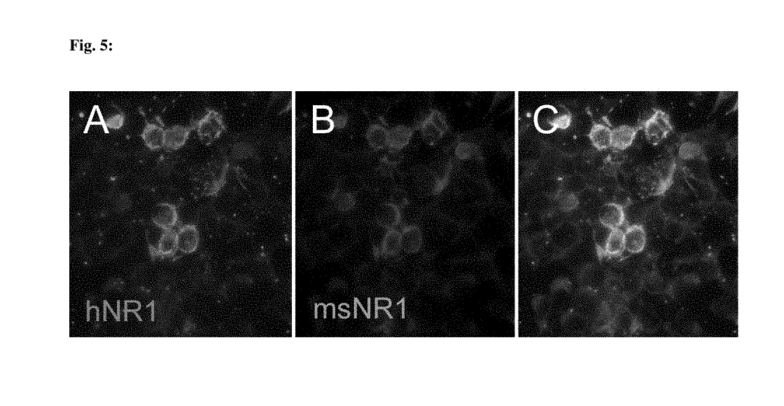

First monoclonal recombinant NMDA1R autoantibody

Staining of NR1-transfected HEK293 cells (diagnostic routine assay) confirming NMDAR-specific binding. (A) hNR1=human monoclonal anti-NR1 antibody, (B) msNR1=commercial mouse anti-NR1 antibody, (C) merged image demonstrating complete staining overlap.

FIG. 6:

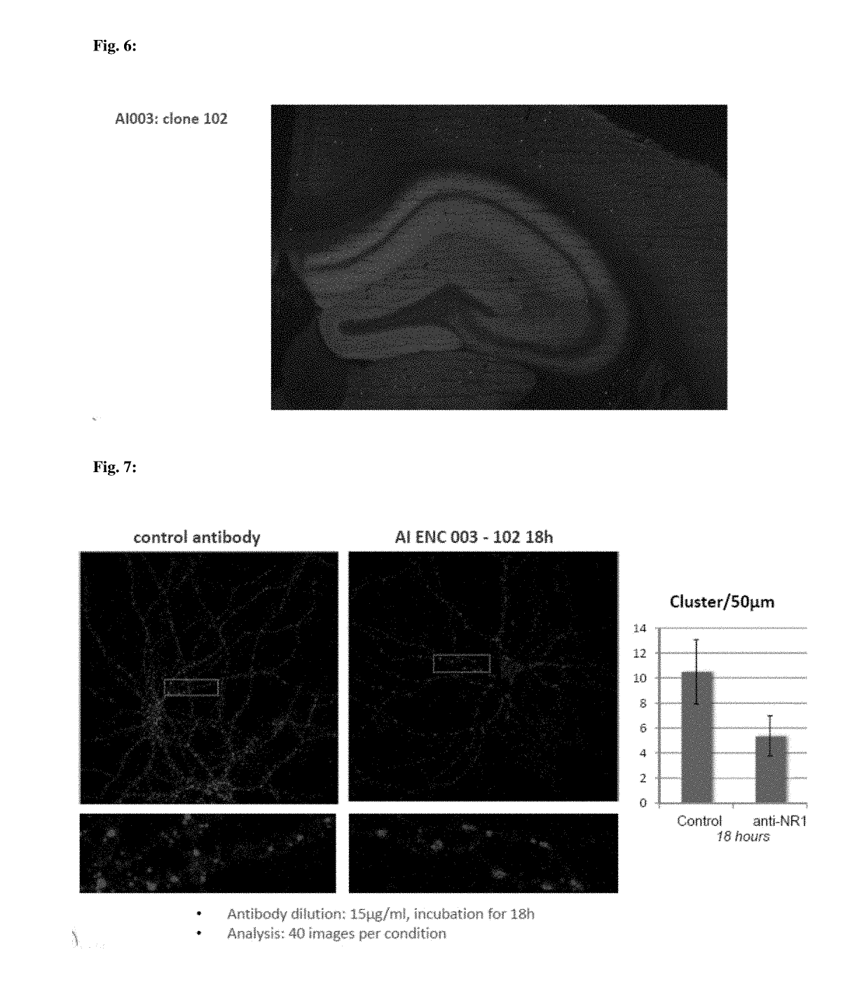

First monoclonal recombinant NMDAR autoantibody.

Specific staining of hippocampus on mouse brain section.

FIG. 7:

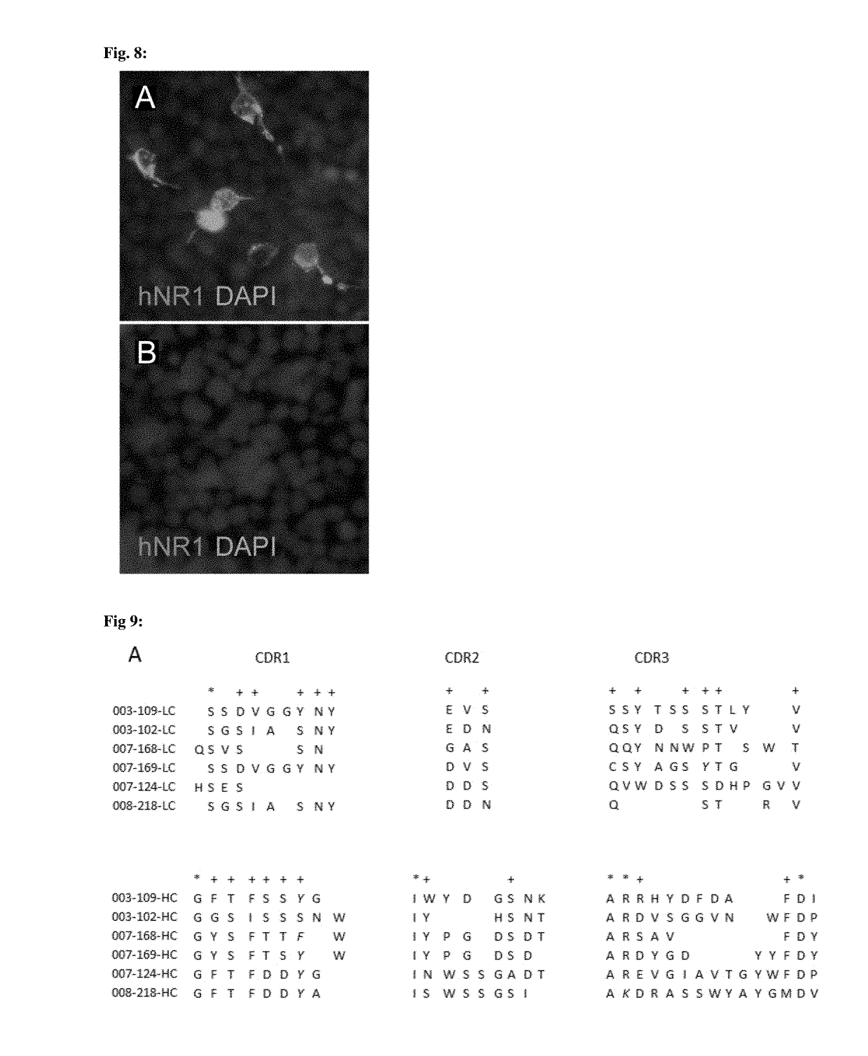

First monoclonal recombinant NMDAR autoantibody.

NMDAR cluster downregulation in hippocampal primary neurons.

FIG. 8:

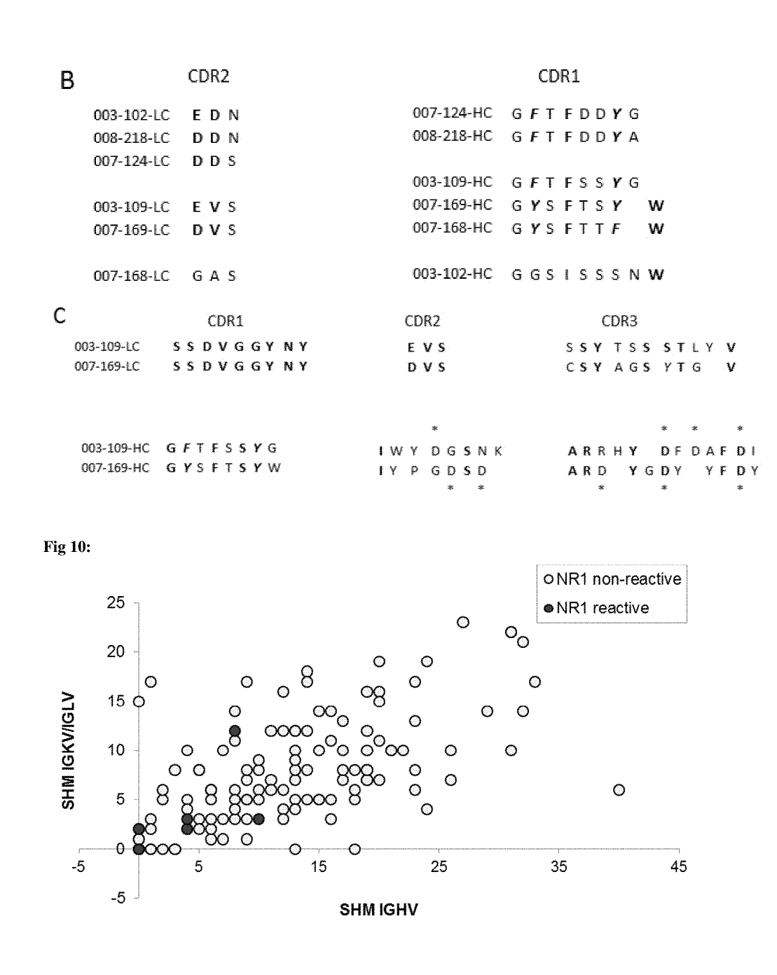

Epitope analysis of monoclonal human NMDAR1 antibodies

HEK293 cells were transfected with wildtype NR1 or a construct with mutation N368Q. As exemplarily shown for clone 007-168, all human monoclonal NMDAR1 antibodies strongly recognized wildtype NR1 (A), but staining of the mutant was eliminated (B).

FIG. 9:

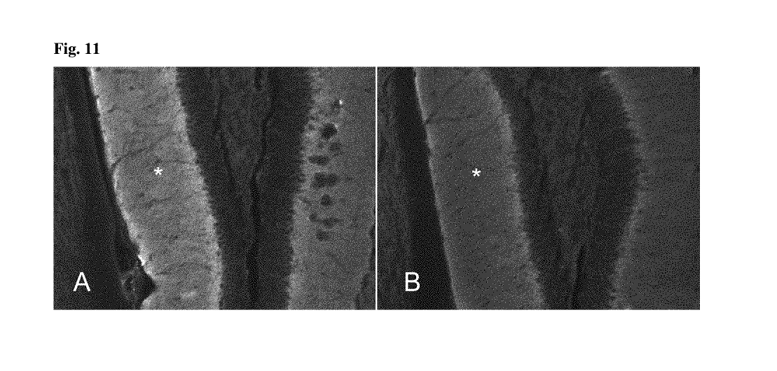

CDR sequence comparison of human monoclonal NMDAR1 antibodies

CDR sequence alignment of human NMDAR antibodies from different patients reveals functional homology. Sequence annotation: * identity in 6/6 sequences; +identity or functional similarity in 5/6 sequences (A). Cross sequence similarities. L CDR2 (right): only 3 residues short=low sterical freedom (in 6/6); dominated by acidic residues (marked in bold (D,E), in 5/6). H CDR1 (left): similar length=8 or 9 residues (in 6/6); dominant aromatic residues (marked in bold (F, Y, W; in 5/6); grouped according to homology (B). NMDAR antibodies derived from different patients show high degree of homology. Comparison of CDR sequences of 003-109 and 007-169. Identity in L CDR1; homology in L CDR2, L CDR3 and H CDR1; similar acidic character in H CDR2 and H CDR3 (C).

FIG. 10:

Number of somatic hypermutations in recombinant human monoclonal NMDAR antibodies. For each generated antibody from antibody-secreting cells in the cerebrospinal fluid of encephalitis patients, the number of somatic hypermutations (SHM) for the V gene segments in the Ig heavy (IGH) and also of the corresponding Ig kappa (IGK) or lambda (IGL) light chains are plotted. The NR1-reactive antibodies (dark dots) show an average of 4.9 SHM in the IGHV and 4.1 SHM in the IGKV/IGLV gene segment which is much less in other (non-NR1) antibodies. Importantly, some NR1 antibodies have not a single hypermutation, thus reflecting naturally occurring antibodies.

FIG. 11:

Aptamers reduce the binding of monoclonal human NMDAR antibodies to mouse brain sections.

Monoclonal NMDAR antibodies strongly bind to the NMDAR-expressing areas in the murine hippocampus (the asterisk marks the dentate gyrus of the hippocampus which shows highest NMDAR protein expression) (A). Preincubation of the same antibodies with the enriched aptamer pool resulted in a marked reduction of antibody binding to the mouse brain (B).

1. Antibody or antibody fragment or non-Ig scaffold binding specifically to a binding region of an anti-NMDAR1 antibody wherein the binding region of said anti-NMDAR1 antibody is comprised in one ore more sequences wherein said one or more sequence is selected from a group consisting of the following sequences:
(003-109-HC)
SEQ ID NO: 1
VQLVESGGGVVQPGRSLRLSCAASGFTFSSYGMHWVRQAPGKGLEWVAV
IWYDGSNKYYADSVKGRFTISRDNSKNTLYLQMNSLRAEDTAVYYCARR
HYDFDAFDIWGQGTMVTVSS
(003-109-LC)
SEQ ID NO: 2
QSALTQPASVSGSPGQSITISCTGTSSDVGGYNYVSWYQQHPGKAPKLM
IYEVSNRPSGVSNRFSGSKSGNTASLTISGLQAEDEADYYCSSYTSSST
LYVFGTGTKVTVL
(003-102-HC)
SEQ ID NO: 3
QVQLQESGPGLVKPSGTLSLTCAVSGGSISSSNWWSWVRQPPGKGLEWI
GEIYHSGNTNYNPSLKSRVTVSVDKSKNQFSLKLTSVTAADTAVYYCAR
DVSGGVNWFDPWGQGTLVTVSS
(003-102-LC)
SEQ ID NO: 4
NFMLTQPHSVSESPGKTVTISCTRSSGSIASNYVQWYQQRPGSAPTTVI
YEDNQRPSGVPDRFSGSIDSSSNSASLTISGLKTEDEADYYCQSYDSST
VVFGGGTKLTVL
(007-168-HC)
SEQ ID NO: 5
VQLVQSGAEAKKPGESLKISCKASGYSFTTFWIGWVRQMPGSGLEWIGI
IYPGDSDTRYSPSFQGHVTISADRSTSTAYLQWSSLKASDTAMYYCARS
AVFDYWGQGTLVTVSS
(007-168-LC)
SEQ ID NO: 6
EIVMTQSPATLSVSPGGRATLSCRASQSVSSNLAWYQQKPGQAPRLLIY
GASTRATGIPVRFSGSGSGTEFTLTISSLQSEDFAVYYCQQYNNWPTSW
TFGQGTKVEIK
(007-169-HC)
SEQ ID NO: 7
EVQLVQSGAEVKKPGESLKISCKGSGYSFTSYWIGWVRQMPGKGLEWMG
IIYPGDSDTRYSPSFQGQVTISADKSISTAYLQWSSLKASDTAMYYCAR
DYGDYYFDYWGQGTLVTVSS
(007-169-LC)
SEQ ID NO: 8
QSALTQPRSVSGSPGQSVTISCTGTSSDVGGYNYVSWYQQHPGKAPKLM
IYDVSKRPSGVPDRFSGSKSGNTASLTISGLQAEDEADYYCCSYAGSYT
GVFGEGTKLTVL
(007-124-HC)
SEQ ID NO: 9
EVQLVESGGGVGRPGGSLRLSCAASGFTFDDYGMSWVRQVPGKGLEWVS
GINWSGADTGYADSVKGRFTISRDNAKNSLYLQMNSLRVEDTALYHCAR
EVGIAVTGYWFDPWGQGTLVTV
(007-124-LC)
SEQ ID NO: 10
SYELTQPPSVSVAPGQTARISCGGNHSESVHWYQQKPGQAPVLVVYDDS
DRPSGIPERFSGSKSGNTATLTISRVEGGDEAEYYCQVWDSSSDHPGVV
FGGGTKLTVL
(007-142-HC)
SEQ ID NO: 11
EVQLVQSGAEVKKPGESLKISCKGSGYSFTSYWIGWVRQMPGKGLEWMG
IIYPGDSDTRYSPSFQGQVTISADKSISTAYLQWSSLKASDTAMYYCAR
DYGDYYFDYWGQGTLVTVSS
(007-142-LC)
SEQ ID NO: 12
LTQPRSVSGSPGQSVTISCTGTSSDVGGYNYVSWYQQHPGKAPKLMIYD
VSKRPSGVPDRFSGSKSGNTASLTISGLQAEDEADYYCCSYAGSYTGVF
GGGTKLTVL
(008-218-HC)
SEQ ID NO: 13
EVQLVESGGGLVQPGRSLRLSCAASGFTFDDYAMHWVRQVPGKGLEWVS
GISWSSGSIGYADSVKGRFTISRDNAKNSLYLQMNSLRAEDTALYYCAK
DRASSWYAYGMDVWGQGTLVTV
(008-218-LC)
SEQ ID NO: 14
NFMLTQPHSVSESPGKTVTISCTRSSGSIASNYVQWYQQRPGSSPTTVI
YDDNQRPSGVPNRFSGSIDSSSNSASLIISGLKTEDEADYYCQSTRVFG
GGTKLTVL
and wherein said binding region for the antibody or antibody fragment or non-Ig scaffold comprises or consists of one or several of the below mentioned sequences:

| | |
|---|---|
| SEQ ID NO: 15 | GFTFSSYG |
| SEQ ID NO: 16 | IWYDGSNK |
| SEQ ID NO: 17 | ARRHYDFDAFDI |
| SEQ ID NO: 18 | SSDVGGYNY |
| SEQ ID NO: 19 | EVS |
| SEQ ID NO: 20 | SSYTSSSTLYV |
| SEQ ID NO: 21 | GGSISSSNW |
| SEQ ID NO: 22 | IYHSGNT |
| SEQ ID NO: 23 | ARDVSGGVNWFDP |
| SEQ ID NO: 24 | SGSIASNY |
| SEQ ID NO: 25 | EDN |
| SEQ ID NO: 26 | QSYDSSTVV |
| SEQ ID NO: 27 | GYSFTTFW |
| SEQ ID NO: 28 | IYPGDSDT |
| SEQ ID NO: 29 | ARSAVFDY |
| SEQ ID NO: 30 | QSVSSN |
| SEQ ID NO: 31 | GAS |
| SEQ ID NO: 32 | QQYNNWPTSWT |
| SEQ ID NO: 33 | GYSFTSYW |
| SEQ ID NO: 34 | IYPGDSD |
| SEQ ID NO: 35 | ARDYGDYYFDY |
| SEQ ID NO: 36 | SSDVGGYNY |
| SEQ ID NO: 37 | DVS |
| SEQ ID NO: 38 | CSYAGSYTGV |
| SEQ ID NO: 39 | GFTFDDYG |
| SEQ ID NO: 40 | INWSGADT |
| SEQ ID NO: 41 | AREVGIAVTGYWFDP |
| SEQ ID NO: 42 | HSES |
| SEQ ID NO: 43 | DDS |
| SEQ ID NO: 44 | QVWDSSSDHPGVV |
| SEQ ID NO: 45 | GYSFTSYW |
| SEQ ID NO: 46 | IYPGDSDT |
| SEQ ID NO: 47 | ARDYGDYYFDY |
| SEQ ID NO: 48 | SSDVGGYNY |
| SEQ ID NO: 49 | DVS |
| SEQ ID NO: 50 | CSYAGSYTGV |
| SEQ ID NO: 51 | GFTFDDYA |
| SEQ ID NO: 52 | ISWSSGSI |
| SEQ ID NO: 53 | AKDRASSWYAYGMDV |
| SEQ ID NO: 54 | SGSIASNY |
| SEQ ID NO: 55 | DDN |
| SEQ ID NO: 56 | QSTRV |

2. Antibody or antibody fragment or non-Ig scaffold binding to a binding region of an anti-NMDAR1 antibody according to claim 1 wherein said antibody or antibody fragment or non-Ig scaffold binding to a binding region of an anti-NMDAR1 antibody is a non-IgG scaffold.

3. Antibody or antibody fragment or non-Ig scaffold binding to a binding region of an anti-NMDAR1 antibody according to claim 1 wherein said non-Ig scaffold may be selected from the group comprising tetranectin-based non-Ig scaffold, fibronectin scaffold, lipocalin-based scaffold, ubiquitin scaffolds, transferrin scaffolds, protein A scaffolds, ankyrin repeat based scaffolds, microproteins, preferably microproteins forming a cysteine knot, scaffolds, Fyn SH3 domain based scaffolds, EGFR-A-domain based scaffolds and Kunitz domain based scaffolds and aptamers.

4. Non-Ig scaffold binding to a binding region of an anti-NMDAR1 antibody according to claim 3 wherein said non-Ig scaffold is an oligonucleotide aptamer.

5. Antibody or antibody fragment or non-Ig scaffold binding to a binding region of an anti-NMDAR1 antibody according claim 1 wherein said antibody or antibody fragment or non-Ig scaffold exhibits an affinity towards said binding region of an anti-NMDAR1 antibody in such that the dissociation constant KD is lower than 10-7 M, preferred 10-8 M, preferred KD is lower than 10-9 M, most preferred lower than 10-10 M. to said binding region of the anti-NMDAR1 antibody.

6. Antibody or antibody fragment or non-Ig scaffold binding to a binding region of an anti-NMDAR1 antibody according to claim 1 wherein said antibody or antibody fragment or non-Ig scaffold neutralizes the anti-NMDAR1 antibodies.

7. Antibody or antibody fragment or non-Ig scaffold binding to a binding region of an anti-NMDAR1 antibody according to claim 1 wherein said antibody or antibody fragment or non-Ig scaffold is a monospecific antibody or antibody fragment or non-Ig scaffold.

8. Antibody or antibody fragment or non-Ig scaffold binding to a binding region of an anti-NMDAR1 antibody according to claim 1 for use in therapy of a disease or a condition in a subject in need of such a therapy said disease or condition being associated with anti-NMDAR1 antibodies.

9. Antibody or antibody fragment or non-Ig scaffold binding to a binding region of an anti-NMDAR1 antibody according to claim 1 for use in therapy of a disease or a condition said disease or condition being associated with anti-NMDAR1 antibodies in a subject in need if such a therapy wherein a subject is in need of such a therapy if said subject exhibits the presence of anti-NMDAR1 antibodies in a bodily fluid and exhibits at least one clinical symptom or clinical condition selected from the group comprising the clinical symptoms/conditions according to the following list (ICD numbers in parentheses refer to the WHO International Classification of Diseases which defines the clinical conditions):
psychiatric abnormalities including depression (F32), mania with psychotic symptoms (F30.2), anxiety (F06.4), phobic anxiety (F40), delusions (F22.0), obsessive-compulsive disorder (F42), organic delusional disorder (F06.3), catatonia (F06.1, F20.2), acute polymorphic psychotic disorder (F23.0, F23.1), dissociative disorders (F44)
movement disorders including dyskinesias/dystonia (G24), myoclonus (G25.3), tremor (G25.0, G25-1, G25-2), tics (F95, G25.69)
epileptic seizures (G40)
hypoventilation (R06.89)
mild cognitive impairment (F06.7)
dementia in Alzheimer's disease (F00), vascular dementia (F01), dementia in other diseases (F02)
pregnancy.

10. Antibody or antibody fragment or non-Ig scaffold binding to a binding region of an anti-NMDAR1 antibody according to claim 1 for use in therapy of a disease or a condition in a subject said disease or condition being associated with anti-NMDAR1 antibodies wherein said antibody or antibody fragment or non-Ig scaffold is administered in vivo to said subject in need thereof.

11. Antibody or antibody fragment or non-Ig scaffold binding to a binding region of an anti-NMDAR1 antibody according to claim 1 for use in therapy of a disease or a condition in a subject said disease or condition being associated with anti-NMDAR1 antibodies wherein said antibody or antibody fragment or non-Ig scaffold is administered to said subject in need thereof intravenously.

12. Antibody or antibody fragment or non-Ig scaffold binding to a binding region of an anti-NMDAR1 antibody according to claim 1 for use in therapy of a disease or a condition in a subject said disease or condition being associated with anti-NMDAR1 antibodies said antibody or antibody fragment or non-Ig scaffold is used in an ex vivo therapy of said subject in need thereof.

13. Antibody or antibody fragment or non-Ig scaffold binding to a binding region of an anti-NMDAR1 antibody according to claim 1 for use in therapy of a disease or a condition in a subject said disease or condition being associated with anti-NMDAR1 antibodies wherein said subject exhibits the presence of anti-NMDAR1 antibodies when measured according to a method of Example 1.2.

14. Antibody or antibody fragment or non-Ig scaffold binding to a binding region of an anti-NMDAR1 antibody according to claim 1 for use in therapy of a disease or a condition in a subject said disease or condition being associated with anti-NMDAR1 antibodies wherein said antibody or antibody fragment or non-Ig scaffold is to be used in combination with a chemotherapeutic agent or a immunosuppressive agent.

15. A method of treatment or prevention of a disease or medical condition associated with anti-NMDAR1 antibodies, which comprises the administration of an effective amount of antibody or antibody fragment or non-Ig scaffold binding to a binding region of an anti-NMDAR1 antibody according claim 1 to a subject in need thereof.

16. A medicament for the treatment or prevention of a disease or medical condition associated with anti-NMDAR1 antibodies comprising a compound according to claim 1.

17. Antibody or antibody fragment or non-Ig scaffold-coated device for plasma exchange or CSF exchange (liquorpheresis) wherein said antibody or antibody fragment or non-Ig scaffold is an antibody or antibody fragment or non-Ig scaffold according to claim 1.

18. Antibody or antibody fragment or non-Ig scaffold-coated device for plasma exchange or CSF exchange (liquorpheresis) according to claim 17 wherein said antibody or antibody fragment or non-Ig scaffold-coated device is an antibody or antibody fragment or non-Ig scaffold-coated column.

19. Use of an antibody or antibody fragment or non-Ig scaffold according to claim 1 for determining the presence of anti-NMDAR1 antibodies in a sample of a bodily fluid of a subject having a disease or condition being associated with anti-NMDAR1 antibodies.

20. A method of determining the presence of an anti-NMDAR1 antibody in a sample of bodily fluid of a subject in order to determine whether said subject is in need of a therapy wherein said subject has a disease or condition being associated with anti-NMDAR1 antibodies and the method comprising:
contacting a sample of bodily fluid obtained from said subject with at least one antibody or antibody fragment or non-Ig scaffold according to claim 1
determining the presence of said anti-NMDAR1 antibodies in said sample of bodily fluid
wherein in case an anti-NMDAR1 antibody is present in said sample said subject may have a disease or condition associated with anti-NMDAR1 antibodies and may be in need of a therapy.

21. Kit for determining the presence of antibodies in sample of a subject that may be in need of a therapy comprising:
1) A solid support with immobilized mixture of NMDAR1-binding antibodies or NMDAR1-binding antibody fragments or NMDAR1-binding non-Ig scaffolds
2) Recombinant human NMDAR1 antibody for determination of standard curve. Anti-human immunoglobulin antibody conjugated to a quantifiable marker
